(19) United States
(12) Patent Application Publication
(10) Pub. No.: US 2024/0077506 A1
(43) Pub. Date: Mar. 7, 2024

(54) SYSTEMS AND METHODS FOR PROCESSING A SAMPLE

(71) Applicant: Sigenex Inc., San Francisco, CA (US)

(72) Inventors: Tanvir Rasab, London (GB); Arnosh Bodhanwala, Bom (IN)

(21) Appl. No.: 18/296,573

(22) Filed: Apr. 6, 2023

Related U.S. Application Data
(63) Continuation of application No. PCT/US2021/053842, filed on Oct. 6, 2021.

(30) Foreign Application Priority Data
Oct. 7, 2020 (GB) 2015911.7

(51) Int. Cl. G01N 35/00; B01L 3/00; G01N 21/03; G01N 21/31; G06Q 10/06; G16H 10/40

(52) U.S. Cl. CPC G01N 35/00069; B01L 3/502715; B01L 3/50273; G01N 21/0303; G01N 21/31; G01N 35/0095; G06Q 10/06; G16H 10/40; B01L 2200/027; B01L 2200/028; B01L 2200/18; B01L 2300/0803; B01L 2300/0816; G01N 2035/00366

(57) ABSTRACT

The present disclosure provides systems, devices, and methods for processing a sample. Systems for processing a sample may include one or more of input modules, processing modules, thermal modules, detection modules, or computer processors. Devices for processing a sample may include cartridges, chips, fluidic pathways, or electronic circuitry. Methods for processing a sample may include one or more of generation of sampling instructions or processing instructions, sample collection, analyte detection, sample analysis, and results reporting.

### CROSS-REFERENCE

This application is a continuation application of International Patent Application No. PCT/US2021/053842, filed Oct. 6, 2021, which claims the benefit of British Patent Application No. GB2015911.7, filed Oct. 7, 2020, each of which is incorporated by reference herein in its entirety.

### BACKGROUND

Personalized or precision medicine is predicted to make a significant change to health services, permitting a movement from one-size-fits all to individualized and targeted treatments for patients. Individualized and targeted treatments may save lives, reduce adverse reactions, and reduce costs. Molecular diagnostics may be a key enabler for the integration of personalized medicine into health care systems. However, few molecular diagnostics are used in clinical practice and access to these technologies remains unequal across countries and sometimes even within individual countries. Many areas of the world have little or no molecular diagnostics capability outside of research labs. In a situation when a patient can afford the costs, samples may be sent to a few centralized or overseas labs, increasing costs and turn-around times for sample processing and analysis. Additionally, many countries prohibit out of country sample processing, making molecular diagnostics inaccessible to large numbers of people. In countries with molecular diagnostic capabilities, the current model is to use expensive centralized labs. Centralized labs may have little automation, no standardization, and manual analysis which may lead to long turn-around times, a need for specifically skilled staff, variability in results, and higher costs. As such, there is a demand for systems, devices, and methods capable of meeting the demand for personalized or precision medicine.

### SUMMARY

The present disclosure provides systems, methods, and devices for sample processing and analysis. The present disclosure provides systems, methods, and devices that enable precise control of micro volumes for sample processing and analysis, which may enable integration of personalized or precision medicine into health care systems to improve diagnostic, reduce costs, and improve health outcomes.

In an aspect, the present disclosure provides a system for processing a sample, comprising: an input module configured to accept one or more chips from a user, wherein at least one of the one or more chips comprises the sample; a processing module configured to transfer the one or more chips from the input module to one or more cartridges, wherein the one or more cartridges are configured to process the sample; and one or more computer processors operatively coupled to the input module and the processing module, wherein the one or more computer processors are individually or collectively programmed to (i) direct the processing module to transfer the one or more chips from the input module to the one or more cartridges and (ii) direct the one or more cartridges to process the sample.

In some embodiments, the system is configured as a closed system. In some embodiments, the system further comprises an alert module configured to monitor the system. In some embodiments, the alert module alerts a user if the processing module is operating outside of set operating parameters. In some embodiments, the alert module is configured to alert a user if the system has been opened or otherwise tampered with. In some embodiments, the system further comprises a tracking module configured to track the sample as it is processed by the system. In some embodiments, the one or more chips or the one or more cartridges comprise (i) at least one machine readable identifier configured to be tracked by the tracking module as the sample is processed by the system or (ii) at least one human readable identifier.

In some embodiments, the system further comprises a sequencing unit configured to sequence at least a portion of the sample. In some embodiments, the system further comprises one or more detection modules configured to analyze the sample. In some embodiments, the system is coupled to a cloud server, and wherein the cloud server is configured to permit a user of the system to remotely monitor and control the system. In some embodiments, the processing module comprises one or more components selected from the group consisting of a liquid handling unit, a pneumatics unit, a temperature control unit, and a transfer unit. In some embodiments, the processing module is configured to transfer the sample from a first chip of the one or more chips to a second chip of the one or more chips. In some embodiments, the processing module is configured to (i) transfer a chip of the one or more chips from a first cartridge of the one or more cartridges to a second cartridge of the one or more cartridges or (ii) transfer the sample from the chip to one or more other chips. In some embodiments, the one or more chips comprise a plurality of ports sealed with a self-sealing material. In some embodiments, the one or more chips are configured to be fluidically connected using disposable connectors configured to puncture the self-sealing material.

In another aspect, the present disclosure provides a method for processing a sample, comprising: providing a system comprising an input module that accepts one or more chips from a user and a processing module that transfers the one or more chips from the input module to one or more cartridges; providing one or more chips to the input module, wherein at least one of the one or more chips comprises the sample; using the processing module to transfer the one or more chips from the input module to one or more cartridges; and sing the one or more cartridges to process the sample.

In some embodiments, the system is configured as a closed system. In some embodiments, the method further comprises using an alert module to monitor the system. In some embodiments, the alert module alerts a user if the processing module is operating outside of set operating parameters. In some embodiments, the alert module alerts a user if the system has been opened or otherwise tampered with. In some embodiments, the method further comprises using a tracking module to track the sample as it is processed by the system. In some embodiments, the one or more chips or the one or more cartridges comprise (i) at least one machine readable identifier that is tracked by the tracking module as the sample is processed by the system or (ii) at least one human readable identifier.

In some embodiments, the method further comprises using a sequencing unit to amplify or sequence at least a portion of the sample. In some embodiments, the method further comprises using one or more detection modules to analyze the sample. In some embodiments, the method further comprises using a cloud server coupled to the system to remotely monitor and control the system. In some embodiments, the processing module comprises one or more components selected from the group consisting of a liquid handling unit, a pneumatics unit, a temperature control unit, and a transfer unit. In some embodiments, the processing module (i) transfers a chip of the one or more chips from a first cartridge of the one or more cartridges to a second cartridge of the one or more cartridges or (ii) transfers the sample from the chip to one or more other chips. In some embodiments, the one or more chips comprise a plurality of ports sealed with a self-sealing material. In some embodiments, the one or more chips are fluidically connected using disposable connectors that puncture the self-sealing material.

In another aspect, the present disclosure provides a system for analyzing a sample, comprising: a processing module configured to couple to one or more cartridges, wherein, during use, a cartridge of the one or more cartridges comprises at least a portion of the sample; a detection module comprising at least one sensor configured to analyze the at least the portion of the sample, wherein the at least one sensor comprises a variable pathlength spectrophotometer configured to be in optical communication with at least a portion of the cartridge comprising the at least the portion of the sample; and one or more computer processors operatively coupled to the processing module and the sensing module, wherein the one or more computer processors are individually or collectively programmed to (i) direct the processing module to couple to the one or more cartridges, (ii) direct the detection module to be in optical communication with the at least the portion of the cartridge comprising the at least the portion of the sample, and (iii) direct the detection module to analyze the sample.

In some embodiments, a pathlength of the variable pathlength spectrophotometer is configured to vary from about 0.01 millimeters (mm) to 0.2 millimeters. In some embodiments, the system is configured as a closed system. In some embodiments, the system further comprises an alert module configured to monitor the system. In some embodiments, the alert module alerts a user if the processing module is operating outside of set operating parameters. In some embodiments, the alert module is configured to alert a user if the system has been opened or otherwise tampered with. In some embodiments, the system further comprises a tracking module configured to track the sample as it is processed by the system. In some embodiments, the one or more chips or the one or more cartridges comprise (i) at least one machine readable identifier configured to be tracked by the tracking module as the sample is processed by the system or (ii) at least one human readable identifier.

In some embodiments, the system further comprises a sequencing unit configured to sequence at least a portion of the sample. In some embodiments, the system further comprises one or more detection modules configured to analyze the sample. In some embodiments, the system is coupled to a cloud server, and wherein the cloud server is configured to permit a user of the system to remotely monitor and control the system. In some embodiments, the processing module comprises one or more components selected from the group consisting of a liquid handling unit, a pneumatics unit, a temperature control unit, and a transfer unit. In some embodiments, the one or more chips comprise a plurality of ports sealed with a self-sealing material. In some embodiments, the one or more chips are configured to be fluidically connected using disposable connectors configured to puncture the self-sealing material.

In another aspect, the present disclosure provides a method for analyzing a sample, comprising: providing a system comprising a processing module that couples to one or more cartridges and a detection module that analyzes the sample; coupling the processing module to the one or more cartridges, wherein a cartridge of the one or more cartridges comprises at least a portion of the sample; positioning the detection module comprising a variable pathlength spectrophotometer in optical communication with at least a portion of the cartridge comprising the at least the portion of the sample; and using the detection module to analyze the at least the portion of the sample.

In some embodiments, a pathlength of the variable pathlength spectrophotometer varies from about 0.01 millimeters (mm) to 0.2 millimeters. In some embodiments, the system is configured as a closed system. In some embodiments, the method further comprises using an alert module to monitor the system. In some embodiments, the alert module alerts a user if the processing module is operating outside of set operating parameters. In some embodiments, the alert module alerts a user if the system has been opened or otherwise tampered with. In some embodiments, the method further comprises using a tracking module to track the sample as it is processed by the system. In some embodiments, the one or more chips or the one or more cartridges comprise (i) at least one machine readable identifier that is tracked by the tracking module as the sample is processed by the system or (ii) at least one human readable identifier.

In some embodiments, the method further comprises using a sequencing unit to amplify or sequence at least a portion of the sample. In some embodiments, the method further comprises using one or more detection modules configured to analyze the sample. In some embodiments, the method further comprises using a cloud server coupled to the system to remotely monitor and control the system. In some embodiments, the processing module comprises one or more components selected from the group consisting of a liquid handling unit, a pneumatics unit, a temperature control unit, and a transfer unit.

In an aspect, the present disclosure provides a system for controlling a temperature of a sample, comprising: a processing module configured to couple to one or more cartridges, wherein, during use, a cartridge of the one or more cartridges comprises at least a portion of the sample; a thermal module configured to control a temperature of at least a portion of the cartridge of the one or more cartridges comprising the at least the portion of the sample, wherein the thermal module comprises a pneumatic temperature control unit configured to be in thermal communication with the at least the portion of the cartridge; and one or more computer processors operatively coupled to the processing module and the thermal module, wherein the one or more computer processors are individually or collectively programmed to (i) direct the processing module to couple to the one or more cartridges, (ii) direct the thermal module to be in thermal communication with the at least the portion of the cartridge, and (iii) direct the thermal module to control the temperature of the at least the portion of the cartridge, thereby controlling a temperature of the at least the portion of the sample.

In some embodiments, the thermal module is configured to provide contactless temperature control. In some embodiments, the thermal module comprises a Rank-Hilsch vortex tube. In some embodiments, the system is configured as a closed system. In some embodiments, the system further comprises an alert module configured to monitor the system. In some embodiments, the alert module alerts a user if the processing module is operating outside of set operating parameters. In some embodiments, the alert module is configured to alert a user if the system has been opened or otherwise tampered with. In some embodiments, the system further comprises a tracking module configured to track the sample as it is processed by the system. In some embodiments, the one or more chips or the one or more cartridges comprise (i) at least one machine readable identifier configured to be tracked by the tracking module as the sample is processed by the system or (ii) at least one human readable identifier.

In some embodiments, the system further comprises a sequencing unit configured to sequence at least a portion of the sample. In some embodiments, the system further comprises one or more detection modules configured to analyze the sample. In some embodiments, the system is coupled to a cloud server, and wherein the cloud server is configured to permit a user of the system to remotely monitor and control the system. In some embodiments, the processing module comprises one or more components selected from the group consisting of a liquid handling unit, a pneumatics unit, a temperature control unit, and a transfer unit. In some embodiments, the one or more chips comprise a plurality of ports sealed with a self-sealing material. In some embodiments, the one or more chips are configured to be fluidically connected using disposable connectors configured to puncture the self-sealing material.

In another aspect, the present disclosure provides a method for controlling a temperature of a sample, comprising: providing a system comprising a processing module that couples to one or more cartridges and a thermal module that controls a temperature of the sample; coupling the processing module to the one or more cartridges, wherein a cartridge of the one or more cartridges comprises at least a portion of the sample; positioning the cartridge such that at least a portion of the cartridge is in thermal communication with the thermal module, wherein the thermal module comprises a pneumatic temperature control unit; and using the thermal module to control a temperature of at least the portion of the cartridge, thereby controlling the temperature of the at least the portion of the sample.

In some embodiments, the thermal module provides contactless temperature control. In some embodiments, the thermal module comprises a Rank-Hilsch vortex tube. In some embodiments, the system is configured as a closed system. In some embodiments, the method further comprises using an alert module to monitor the system. In some embodiments, the alert module alerts a user if the processing module is operating outside of set operating parameters. In some embodiments, the alert module alerts a user if the system has been opened or otherwise tampered with. In some embodiments, the method further comprises using a tracking module to track the sample as it is processed by the system. In some embodiments, the one or more chips or the one or more cartridges comprise (i) at least one machine readable identifier that is tracked by the tracking module as the sample is processed by the system or (ii) at least one human readable identifier.

In some embodiments, the method further comprises using a sequencing unit to amplify or sequence at least a portion of the sample. In some embodiments, the method further comprises using one or more detection modules configured to analyze the sample. In some embodiments, the method further comprises using a cloud server coupled to the system to remotely monitor and control the system. In some embodiments, the processing module comprises one or more components selected from the group consisting of a liquid handling unit, a pneumatics unit, a temperature control unit, and a transfer unit.

In another aspect, the present disclosure provides a device for processing a sample, comprising: a cartridge comprising one or more bays, wherein a bay of the one or more bays is configured to removably hold a chip, and wherein the bay comprises a first pattern of contact points, wherein the chip comprises a second pattern of contact points that are complementary to the first pattern of contact points.

In some embodiments, the cartridge is configured such that the bay is configured to removably couple to more than one type of chip. In some embodiments, the chip is configured to be transferable from the cartridge to another cartridge. In some embodiments, the chip is configured to process the sample. In some embodiments, the chip is configured to perform at least one function during processing of the sample. In some embodiments, the at least one function is selected from the group consisting of extracting nucleic acid from the sample, library preparation, sequencing the sample, separating components from the sample, and performing an assay on the sample.

In some embodiments, the cartridge comprises mesofluidic circuitry and macro-sized contacts and the chip comprises microfluidic circuitry and micro-sized contacts, and wherein the first pattern of contact points and the second pattern of contact points are configured to provide an interface between the macro-sized contacts and the micro-sized contacts. In some embodiments, the cartridge comprises a standard interface such that the cartridge is capable of performing more than one type of sample processing. In some embodiments, the cartridge is configured to analyze a sample via exchanging the chip for another chip.

In some embodiments, the cartridge or the chip comprises an authentication unit configured for validation and tracking. In some embodiments, the authentication unit provides cryptographic security. In some embodiments, the cartridge comprises one or more members selected from the group consisting of pneumatics, transducers, actuators, sensors, micropumps, pressure generators, regulators, solenoid valves, electromagnets, temperature sensors, energy storage units, and electronic circuitry. In some embodiments, a contact of the first pattern of contacts and the second pattern of contacts is configured to provide one or more of power, electronic communication, pneumatic communication, electromagnetic communication, or any combination thereof to the chip. In some embodiments, the cartridge comprises a memory and wherein the memory stores an interface protocol or driver for the chip. In some embodiments, the chip comprises a self-sealing material, and wherein the cartridge comprises one or more needles to penetrate the self-sealing material.

In another aspect, the present disclosure provides a device for collecting a sample, comprising: an inlet port configured to collect a sample from a subject; one or more chips in fluid communication with the inlet port; and an adapter in fluid communication with the inlet port and the one or more chips, wherein the adapter is configured to direct the sample from one or more mesofluidic channels of the inlet port to one or more microfluidic channels of the one or more chips.

In some embodiments, the inlet port is fluidically connected to a needle configured to draw blood from a subject. In some embodiments, the inlet port is configured to seal. In some embodiments, the device comprises at least two chips, and wherein the adapter is configured to multiplex the sample into each chip of the at least two chips. In some embodiments, the adapter is configured to transport the sample. In some embodiments, the adapter is configured for one or more of chip detection, chip identification, temperature control, temperature detection, location detection, data logging, tamper detection, or any combination thereof.

In some embodiments, the device is configured to track and monitor the sample once the sample is input into the device. In some embodiments, the device is self-sealing. In some embodiments, a chip of the one or more chips is configured to (i) hold the sample or (ii) provide buffers, reagents, or other additives to the sample.

In another aspect, the present disclosure provides a system for processing a sample of a subject, comprising: a computer server in communication with a plurality of user devices, wherein the computer server comprises a (i) a database for storing test information and clinical information, (ii) a memory for storing a set of software instructions, and (iii) one or more computer processors configured to execute the set of software instructions to: receive, from a first user device, a request for analysis of the sample; request, from a second user device, health or physiological information of the subject; query the database to (i) retrieve the test information and the clinical information and (ii) use the test information and the clinical information to generate pre-collection constraints and a sample collection protocol; provide the pre-collection constraints to a user of the second user device; and provide the sample collection protocol to a third user device, wherein the sample collection protocol permits a sample collector to collect the sample for the test.

In some embodiments, the health or physiological information of the subject is selected from the group consisting of medical history, over the counter medication usage, supplement usage, and combinations thereof. In some embodiments, the system is configured to alert the first user device or the second user device if a medication, over the counter medication, or supplement interferes with the analysis of the sample. In some embodiments, the system is further configured to provide an estimated turnaround time from sample collection to receiving results of the analysis of the sample to the first user device or the second user device. In some embodiments, the system is further configured to provide scheduling information to the first user device, the second user device, or the third user device. In some embodiments, the scheduling information comprises sample collection date, sample collection time, location of sample collection, personnel assigned to collect the sample, or any combination thereof. In some embodiments, the pre-collection constraints comprise dietary requirements or fasting requirements. In some embodiments, the system is configured to provide reminders to the second user device regarding the pre-collection constraints. In some embodiments, the sample collection protocol comprises materials used for sample collection or sample collection workflow. In some embodiments, the system is further configured to prompt a sample collection personnel to positively identify a subject prior to sample collection.

In another aspect, the present disclosure provides a method for processing a sample of a subject, comprising: providing a computer server in communication with a plurality of user devices, wherein the computer server comprises (i) a database for storing a test information and clinical information (ii) a memory for storing a set of software instructions, and (iii) one or more computer processors to execute the set of software instructions; receiving from a first user device of the plurality of user devices a request for analysis of the sample; requesting health or physiological information of the subject from a second user device of the plurality of user devices; querying the database to retrieve the test information and the clinical information; using the test information and the clinical information to generate pre-collection constraints and a sample collection protocol; providing the pre-collection constraints to the second user device; and providing the sample collection protocol to a third user device of the plurality of user devices, wherein the sample collection protocol permits a sample collector to collect the sample of the subject for analysis of the sample.

In some embodiments, the health or physiological information is selected from the group consisting of patent medical history, over the counter medication usage, supplement usage, and combinations thereof. In some embodiments, the method further comprises alerting the first user device or the second user device if a medication, over the counter medication, or supplement interferes with the analysis of the sample. In some embodiments, the method further comprises providing an estimated turnaround time from sample collection to providing results of the analysis of the sample to the first user device or the second user device. In some embodiments, the method further comprises providing scheduling information to the first user device, the second user device, or the third user device. In some embodiments, the scheduling information comprises sample collection date, sample collection time, location of sample collection, personnel assigned to collect the sample, or any combination thereof. In some embodiments, the pre-collection constraints comprise dietary requirements or fasting requirements. In some embodiments, the method further comprises providing reminders to the second user device regarding the pre-collection constraints. In some embodiments, the sample collection protocol comprises materials used for sample collection or sample collection workflow. In some embodiments, the method further comprises prompting a sample collection personnel to positively identify a subject prior to sample collection.

In another aspect, the present disclosure provides a system for analyzing a sample of a subject, comprising: a computer server in communication with a user device and an analysis module configured to analyze the sample, wherein the computer server comprises a (i) a database for storing test information, (ii) a memory for storing a set of software instructions, and (iii) one or more computer processors configured to execute the set of software instructions to: receive, from the user device, one or more input parameters, wherein the one or more input parameters comprise type of analysis and number of tests to be performed; In some embodiments, query the database to determine one or more testing conditions for performing the analysis of the sample; receive, from the analysis module, a status of the analysis module; use the one or more input parameters, the one or more testing conditions, and the status of the analysis module to generate a testing schedule with a minimum testing turnaround time; and provide the testing schedule to the analysis module to perform the analysis of the sample.

In some embodiments, the database further comprises protocols for the analysis, result analysis guidelines, recommendation guidelines, or any combination thereof. In some embodiments, the one or more input parameters further comprises testing urgency. In some embodiments, the one or more testing conditions comprises a number of operations required per assay, common sub-processes shared between assays, or materials required for each assay. In some embodiments, the status of the analysis module comprises capacity of the analysis module, number of chip bays available, current operation status, or estimated time for each test to be run. In some embodiments, the system is configured such that analysis of additional samples is permitted to be added to an analysis queue during operation of the system. In some embodiments, the system is further configured to alert the user device when the analysis of the sample is complete. In some embodiments, the system is further configured to provide results of the analysis to the user device. In some embodiments, the system is further configured to provide results of the analysis to a network of authorized experts, wherein a list of authorized experts is stored in the database. In some embodiments, the network of authorized experts comprises an independent panel of verified experts. In some embodiments, the system is further configured to permit the network of authorized experts to provide recommendations based on the results of the analysis of the sample.

In another aspect, the present disclosure provides a method for analyzing a sample of a subject, comprising: providing a computer server in communication with a user device and an analysis module, wherein the computer server comprises (i) a database for storing test information (ii) a memory for storing a set of software instructions, and (iii) one or more computer processors configured to execute the set of software instructions; receiving one or more input parameters from the user device, wherein the one or more input parameters comprise type of analysis and number of tests to be performed; querying the database to determine one or more testing conditions for performing the analysis of the sample; receiving a status of the analysis module from the analysis module; using the one or more input parameters, the one or more testing conditions, and the status of the analysis module to generate a testing schedule with a minimum testing turnaround time; and providing the testing schedule to the analysis module to perform the analysis of the sample.

In some embodiments, the database further comprises protocols for the analysis, result analysis guidelines, recommendation guidelines, or any combination thereof. In some embodiments, the one or more input parameters further comprises testing urgency. In some embodiments, the one or more testing conditions comprises a number of operations required per assay, common sub-processes shared between assays, or materials required for each assay. In some embodiments, the status of the analysis module comprises capacity of the analysis module, number of chip bays available, current operation status, or estimated time for each test to be run. In some embodiments, the method further comprises permitting additional analytical tests to be added to an analysis testing queue during operation of the system. In some embodiments, the method further comprises alerting the user device when the analysis of the sample is complete. In some embodiments, the method further comprises providing results of the analysis of the sample to the user device. In some embodiments, the method further comprises providing results of the analysis of the sample to a network of authorized experts, wherein a list of the authorized experts is stored in the database. In some embodiments, the network of authorized experts comprises an independent panel of verified experts. In some embodiments, the method further comprises permitting the network of authorized experts to provide recommendations based on the results of the analysis of the sample.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows an example external housing of an integrated system for processing a sample;

FIG. 2 schematically illustrates an example process flow for a sample through the integrated system and consumable inputs;

FIG. 3 schematically illustrates a bench top system couples to multiple user applications and a central data processing network;

FIG. 4 schematically illustrates flow of information, process control data, and sample flow from the various applications, bench top system, and control center;

FIG. 5 schematically illustrates an example process flow for sample ordering and tracking;

FIG. 6 schematically illustrates the database (e.g., knowledge base) and bioinformatics;

FIG. 7 schematically illustrates an example run manifest including scheduled and unscheduled runs along with missing components;

FIG. 8 shows an example process flow for an example autosampler;

FIG. 9 shows example holders with multiple cartridges;

FIG. 10 shows an example cartridge, removable chips, and chip components;

FIG. 11 schematically illustrates an example processing unit comprising multiple arms in communication with example cartridges;

FIG. 12 schematically illustrates actuation of an arm with multiple sensors;

FIGS. 13A and 13B schematically illustrate example analysis systems; FIG. 13A schematically illustrates separate systems configured to each run a set of assays; FIG. 13B schematically illustrates an integrated system configured to run multiple sets of assays;

FIG. 14 shows an example disk shaped cartridge with a plurality of similar chips, a cartridge with different types of chips, and a cartridge with a single chip;

FIG. 15 shows an example cartridge with four chip bays loaded with four different chip types;

FIG. 16 schematically illustrates the compatibility of a given bay with multiple chip types;

FIG. 17 schematically illustrates examples of bay geometries and contact point topologies;

FIG. 18 shows an example of a connector puncturing the self-sealing material of a chip port;

FIG. 19 schematically illustrates a chip with a high density of ports and minimal dead space;

FIG. 20 schematically illustrates an example connector;

FIG. 21 schematically illustrates an example process flow for reverse transcription polymerase chain reaction and next generation sequencing;

FIG. 22 schematically illustrates an example heat exchanger for thermal cycling;

FIG. 23 schematically illustrates an example contactless temperature control system;

FIGS. 24A and 24B schematically illustrate an example of in-plane variable pathlength spectrophotometry;

FIG. 25 schematically illustrates an example of a sample collection device;

FIG. 26 schematically illustrates an example adapter and holder;

FIG. 27 schematically illustrates an example interface between an adapter and multiple chip types;

FIG. 28 shows an example process flow for venipuncture;

FIG. 29 shows an example process flow for venipuncture using a sample collection device;

FIG. 30 schematically illustrates an example laboratory information management system;

FIG. 31 schematically illustrates an example of instrument integration and automation;

FIG. 32 schematically illustrates an example matrix of parameters for sample ordering and scheduling;

FIG. 33 schematically illustrates an example process flow for analyzing a sample and LIMS integration;

FIG. 34 schematically illustrates an example prompt, capture, and review cycle for sample collection;

FIG. 35 schematically illustrates an example process flow of samples for NGS analysis;

FIG. 36 schematically illustrates and example process flow for an example urgent test;

FIG. 37 schematically illustrates example architecture of a knowledge base;

FIG. 38 schematically illustrates an example process flow for the second opinion review network;

FIG. 39 shows an example process flow for the development of assays on the analysis platform; and

FIG. 40 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “mesofluidic,” as used herein, generally refers to fluid handling components on the scale of millimeters to centimeters. For example, a mesofluidic fluid flow path (e.g., channel), chamber, or other fluid handling component may have one or more dimensions (e.g., diameter, depth, etc.) on the order of 1 millimeter (mm), 10 mm, 100 mm, or 1 centimeter (cm). Mesofluidic devices may be configured to or otherwise used to process volumes of fluid on the order of milliliters (mL). For example, a mesofluidic device may process fluid volumes on the order of 1 mL, 10 mL, 100 mL, or more.

The term “microfluidic,” as used herein, generally refers to fluid handling components on the scale of micrometers (μm). For example, a microfluidic fluid flow path (e.g., channel), chamber, or other fluid handling component may have one or more dimensions (e.g., diameter, depth, etc.) on the order of 1 μm, 10 μm, 100 μm, or larger. Microfluidic devices may be configured to or otherwise used to process volumes of fluid on the order of microliters (μL) to picoliters (μL). For example, a microfluidic device may process fluid volumes on the order of 1 μL, 10 μL, 100 μL, 1 μL, 10 μL, 100 μL, or more.

The term “closed system,” as used herein, generally refers to a system configured to analyze a sample with little or no operator interventions. For example, a closed system may not have or permit the transfer of matter in or out of the system by an operator during operation. For example, a closed system may permit transfer of sample or consumables into the system by an operator when the system is not operating (e.g., before operation), but may not permit intervention during sample processing. In another example, the closed system may permit samples, consumables, reagents, etc. to be input into the system before, during, or after operation, but may not permit intervention in the processing of the sample. The closed system may be coupled to one or more other modules or devices (e.g., a sequencer) such that matter (e.g., samples) may be transferred to the coupled modules or devices without human intervention.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Reducing costs for next generation sequencing (NGS) and increasing identification and association of nucleic acid biomarkers for a variety of genetic disorders and clinical diseases (e.g., infectious diseases, antimicrobial resistance, cancer, etc.) has increased the demand for molecular diagnostics that go beyond NGS. However, the demands on lab infrastructure, trained technicians for sample processing, computing infrastructure, and trained bioinformaticians to interpret results from the increased amount data produced by NGS may significantly reduce viability for low volume, point-of-care (POC), or resource limited settings.

Specialized liquid handling robots may provide a degree of automation with high reproducibility and quality output with reduced hands on time (e.g., manual operation). However, the demands on lab infrastructure and trained technicians may not be alleviated. For example, reagents and samples may be aliquoted manually and each instrument may be operated in a contamination free environment. Quality control may be performed on the output data and the results may be interpreted by the technicians before continuing to the next step in the process.

Utilizing a general purpose liquid handler may reduce hands on time (e.g., manual operation), but may require technical knowledge for the operator and may increase environmental and cross contamination risks. Integrating and automating individual devices into a single platform may raise costs and footprint of the device, making them viable for large labs with high volume, high throughput requirements. However, such systems may be unviable for smaller labs with fewer resources.

Reagent kits may be manufactured for batch processing of multiple samples (e.g., 24, 96, 384, etc.) for both manual and automated processing. This may further add to the complexity and cost for low volume applications in a POC setting where the number of each assay type is lower than the batch sizes.

Microfluidic circuits may provide an increased degree of control for small volumes that may not be matched by macrofluidic liquid handling tools. Miniaturization may scale parallelization of heterogeneous processes from low to high throughput at a fraction of the cost and footprint of a macro-scale automation platform. Microfluidic circuits may provide a closed system that may reduce contamination risks and allow for consistent, reproducible results across devices, labs, and operators.

Microfluidic circuits may enable precise control of micro volumes and short fluidic paths may reduce dead volume and optimize reagent and sample utilization. Shortened path lengths, larger surface areas along with functionally optimized circuitry may allow for transfers, mixing and homogenous temperature control at throughput efficiencies superior to equivalent macro-scale processes.

However, the attributes that enable microfluidic circuits to be so efficient may also make the microfluidic circuits specialized. For example, micro total analysis systems may use higher order functionality from individual circuits which may be non-trivial. Due to the tight integration between circuit and instrumentation, modifications may have a cascading effect on design and instrumentation and a significant impact on manufacturing, supply chain, retrofitting and dead inventory. This may reduce the ability to upgrade systems regularly and therefore risk obsolescence, particularly in a nascent field such as molecular diagnostics where improvements to existing protocols and new assays are constantly being generated.

### Systems and Methods for Sample Processing

Instruments that combine the flexibility of a liquid handling robot and the efficiency of microfluidic circuits may enable ‘sample in-results out’ molecular diagnostic capabilities for low volume multi-assay application. Such systems may dramatically reduce costs in terms of consumables, operator training, and infrastructure requirements.

In an aspect, the present disclosure provides systems for processing a sample. The system may include an input module, a processing module, and one or more computer processors operatively coupled to the input module and the processing module. The input module may be configured to or may otherwise accept one or more cartridges from a user. At least one of the one or more chips may include the sample. The processing module may be configured to or may otherwise transfer the one or more chips from the input module to one or more cartridges. The one or more cartridges may be configured to or otherwise process the sample. The one or more computer processors may be individually or collectively programmed to direct the processing module to transfer the one or more chips from the input module to the one or more cartridges and direct the one or more cartridges to process the sample.

In another aspect, the present disclosure provides systems for processing a sample. The system may include a processing module, a detection module, and one or more computer processors operatively coupled to the processing module and the detection module. The processing module may be configured to couple to or may couple to one or more cartridges. During use, a cartridge of the one or more cartridges may comprise at least a portion of the sample (e.g., the sample may be within a chip interfaced with the cartridge). The detection module may include at least one sensor. The sensors may be configured to or may otherwise analyze the portion of the sample in the cartridge. One or more sensors may be a variable pathlength spectrophotometer configured to be in or in optical communication with the portion of the sample in the cartridge. The one or more computer processors may be individually or collectively programmed to direct the processing module to couple to the one or more cartridges, direct the detection module to be in optical communication with the portion of the cartridge with the sample, and direct the detecting module to analyze the sample.

In another aspect, the present disclosure provides a system for processing a sample. The system may include a processing module, a thermal module, and one or more computer processors operatively coupled to the processing module and the thermal module. The processing module may be configured to be or may be coupled to one or more cartridges. During use, the one or more cartridges may comprise at least a portion of the sample. The thermal module may be configured to or may otherwise control a temperature of at least a portion of a cartridge of the one or more cartridges. The thermal module may comprise a pneumatic temperature control unit configured to be in or otherwise in thermal communication with at least a portion of the cartridge. The one or more computer processors may be individually or collectively programmed to direct the processing module to couple to the one or more cartridges, direct the thermal module to be in thermal communication with at least a portion of the cartridge, and direct the thermal module control the temperature of at least a portion of the cartridge to thereby control a temperature of a portion of the sample.

In another aspect, the present disclosure provides methods for processing a sample. Methods may include providing a system comprising an input module and a processing module. The input module may accept one or more chips from a user. A user may provide one or more chips to the input module. A chip may or may not include a sample, reagents, other assay components, or a combination thereof. In an example, at least one of the chips comprises the sample. The processing module may transfer the chips from the input module to one or more cartridges. The cartridges may be input into the system at the input module or at another location. The system may use the one or more cartridges to process the sample.

In another aspect, the present disclosure provides methods for processing a sample. The method may include providing a system that includes a processing module and a detection module. The processing module may couple to one or more cartridges and the detection module may analyze the sample. The processing module may be coupled to the cartridges. At least one of the cartridges may comprise a portion of the sample. The detection module may comprise a variable pathlength spectrophotometer. The system may position the variable pathlength spectrophotometer in optical communication with at least a portion of the cartridge with the sample. The detection module may analyze the portion of the sample.

In another aspect, the present disclosure provides methods for processing a sample. The method may include providing a system that includes a processing module and a thermal module. The processing module may couple to one or more cartridges and the detection module may analyze the sample. The processing module may be coupled to the cartridges. At least one of the cartridges may comprise a portion of the sample. The thermal module may control a temperature of the sample and may comprise a pneumatic temperature control unit. The cartridge may be positioned such that at least a portion of the cartridge is in thermal communication with the thermal module. The thermal module may be used to control the temperature of a portion of the cartridge to thereby control a temperature of the sample or a portion of the sample.

In another aspect, the present disclosure provides devices for processing a sample. The devices may include a cartridge with one or more bays. A bay may be configured to or may otherwise removably hold a chip. They bay (e.g., each bay of the one or more bays) may include a pattern of contact points (e.g., fluidic and electrical contact points). The chip may also include a pattern of contact points which are complementary to the pattern of contact points on the cartridge. The contact points may provide a combination of fluidic, electrical, and thermal interfaces between the chips and the cartridges. The contact point may provide one or more of fluidic, electrical, or thermal interfaces between the chips and the cartridge.

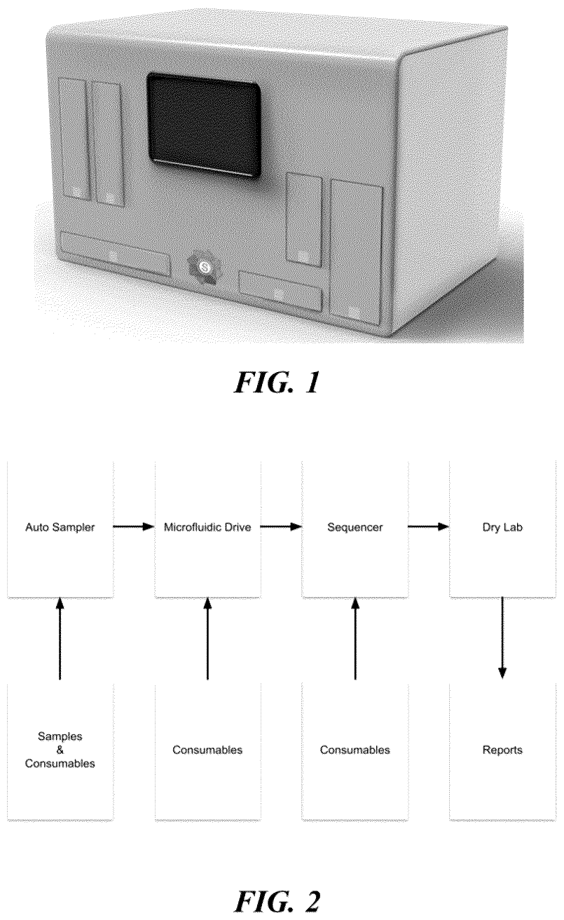

An example of such a system, as shown in FIGS. 1 and 2, may include an autosampler, a microfluidic drive for nucleic acid extraction and library preparation, a sequencer, or a bioinformatics system (e.g., dry lab). FIG. 1 shows an example of an external housing of such a system and FIG. 2 shows an example of inputs to the system and outputs from the system. For example, a sample and consumables may be input into the autosampler. The sample may be transferred via the microfluidic drive to a sequencer. Both the microfluidic drive and sequencer may use various consumables, such as reagents. Results from the sequencer may be provided to a dry lab, which may generate a report. The dry lab may include a dock for storage and application specific hardware. Alternatively, or in addition to, the dry lab may be implemented on a local or offsite server cluster or be cloud based.

The system may have many benefits as compared to other fluid handling and sample analysis systems. In an example, the system is easy to install in that the system may be a self-contained microenvironment such that the system does not use any or uses minimal external infrastructure (e.g., clean rooms or laminar hoods). In another example, the system is easy to operate and operators may have minimal training and may intervene minimally with the system. Operator intervention may be limited to loading samples and consumables into the system. In another example, the system may be easy to maintain in that it may be cloud connected to provide automated updates to firmware, protocols, applications, reference datasets, bioinformatics, or any combination thereof. The system may have active self-diagnostics for fault detection and point of failure identification. Additionally, the system may permit remote troubleshooting and include a predictive module for proactive maintenance. In another example, sample analysis may be easy to interpret due to a curated database (e.g., knowledge base) and artificial intelligence assisted report interpretation. The system may be connected to a network of domain experts to analyze reports, metadata, and/or de-identified data conformant to local regulations for improved insights and faster interventions. In another example, the system may permit rapid testing due to microfluidic manipulation of sample and reagents for wet lab processing and hardware accelerated dry lab pipeline for shortened turnaround times (e.g., within minutes or hours from when an analysis is completed). In another example, the system may include a modular configuration with stackable and swappable components. The stackable and swappable components may permit optimization of each device, run, or both for different workloads, applications, and protocols with minimal downtime between tests. In another example, the system may permit multiplexed assays and extraction and purification from various sample types. The system may permit preparation of different libraries per different assay protocols for the different samples and sequence the samples together in a single run. In another example, the system may be cost effective due to automation of highly technical and labor intensive processes. The reduced cost as compared to other liquid handling and sample analysis platforms may enable sequencing to be brought into the point-of-care (POC) setting. Individual reagent cartridges per chip may optimize consumption and multiplexed assays may be run in any combination and may not be dependent upon batch size of equivalent macro kits.

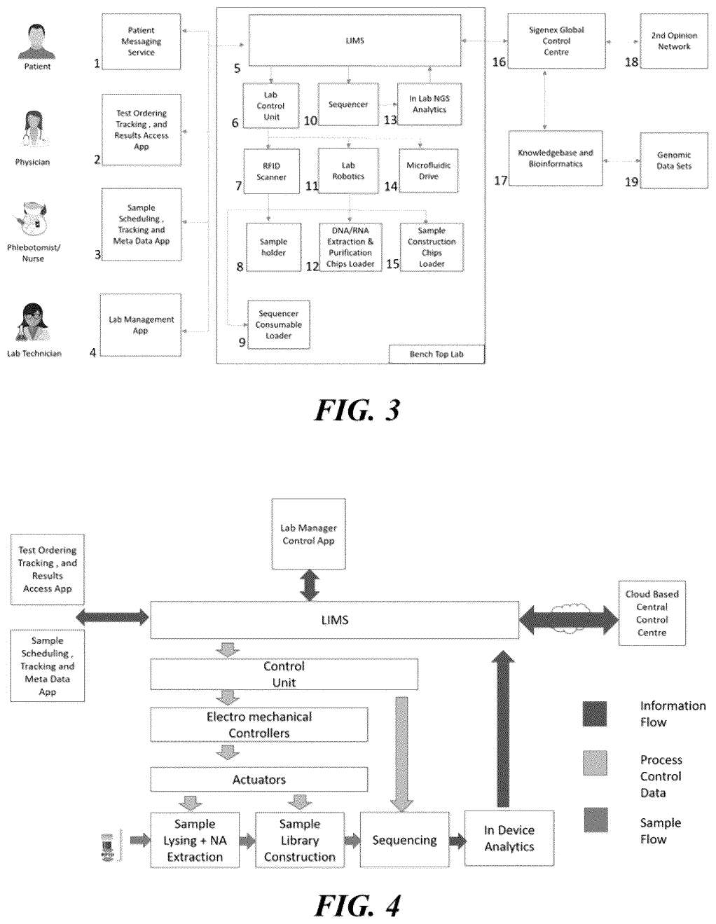

The system may be in communication with or coupled to external user devices, a cloud server, or both external user devices and a cloud server. The cloud server may be configured to or otherwise permit a user of the system to remotely monitor and control the system. FIG. 3 shows an example infrastructure coupling the system to a cloud server. The cloud server may provide access of the system to a patient messaging service 1, test ordering tracking and results access application 2, sample scheduling, tracking, and meta data application 3, a lab management application 4, or any combination thereof. The messaging service for patients may communicate via text, email, or other messaging service or application running on the patient's device to schedule appointments and capture metadata at the time of sample collection. The test ordering application or interface for doctors and/or clinicians may permit the doctor or clinician to examine a patient, select appropriate test, place order for the test through the application or a web interface that is accessed via the application program interface of existing enterprise resource planning system for a medical center. The sample management application for phlebotomists or nurses may permit a sample collector to receive details of the test order, patient name, identification, and other pertinent details, schedule an appointment to collect the sample, and to collect the sample (e.g., blood, plasma, urine, liquefied stool, or any other sample described herein). The sample management application may further be used to collect additional metadata about the patient. The lab management application or web interface may permit a laboratory technician to manage the operations of the laboratory.

The system (e.g., bench top lab) may include the laboratory information management system (LIMS) 5, a lab control unit 6, radiofrequency identifier (RFID) or barcode scanner 7, sample holder 8, sequencer consumable loader 9, sequencer 10, lab robotics 11, nucleic acid extraction and purification chips loader 12, in lab next generation sequencer analytics 13, microfluidic drive 14, sample construction chips loader 15, or any combination thereof. The LIMS may connect (e.g., via the internet, local area network, Wi-Fi, wide area network, etc.) to the different applications, cloud based central management control center, and the internal laboratory control unit. The LIMS may also download different sample construction protocol processes and transfer the relevant process instruction codes to the lab control unit. The laboratory control unit may control the automation, robotics, and electro-mechanical components that carry out the wet laboratory work, such as preparing the samples for sequencing. The RFID scanner or reader may read the sample tube, container, or chip identifications (e.g., extraction purification chips, sample construction chips, etc.). The sample holder and loader may include an electro-mechanical sub system for holding sample tubes, containers, or chips for wet lab processing. The sequencer consumable loader may include an automated mechanism for loading sequencer consumables into the sequencer integrated into the laboratory. The sequencer may be an integrated next generation sequencer (NGS), integrated with and adapted to work with the control unit, robotics, and NGS analytics. The laboratory robotics may be automated robotics that carries out the picking and placing of the chips, loading of the sequencer consumables into the sequencer as well as sequencer chips with the constructed sample libraries. The nucleic acid extraction and purification microfluidic chip holder and loader may be an electro-mechanical subsystem that holds the microfluidic chips for nucleic acid extraction and purification. The processing module (e.g., microfluidic drive) may carry out the wet lab functions from sample lysis, nucleic acid extraction, purification, or any combination thereof. The sample construction chip holder and loader may be an electromechanical subsystem that holds microfluidic chips for sample construction.

The cloud server may further couple the system to a global control center 16, database (e.g., knowledge base) and bioinformatics 17, second opinion network 18, genomic data set 19, or any combination thereof. The global control center may be a cloud based laboratory network management center. The global control center may permit monitoring and operation of the deployed laboratory, receive anonymized test sequenced and personal metadata of the patient, and forward the data to the bioinformatics and data base (e.g., knowledge base) be analyzed further and matched. The database (e.g., knowledge base) and bioinformatics may be an artificial intelligence or machine learning based system that matches the sequenced data with the most relevant genomics data sets, published clinical studies, and published scientific research papers. The platform may permit a network of verified domain experts to give their expert opinion on the results report and recommend actions to the clinician or doctor may follow.

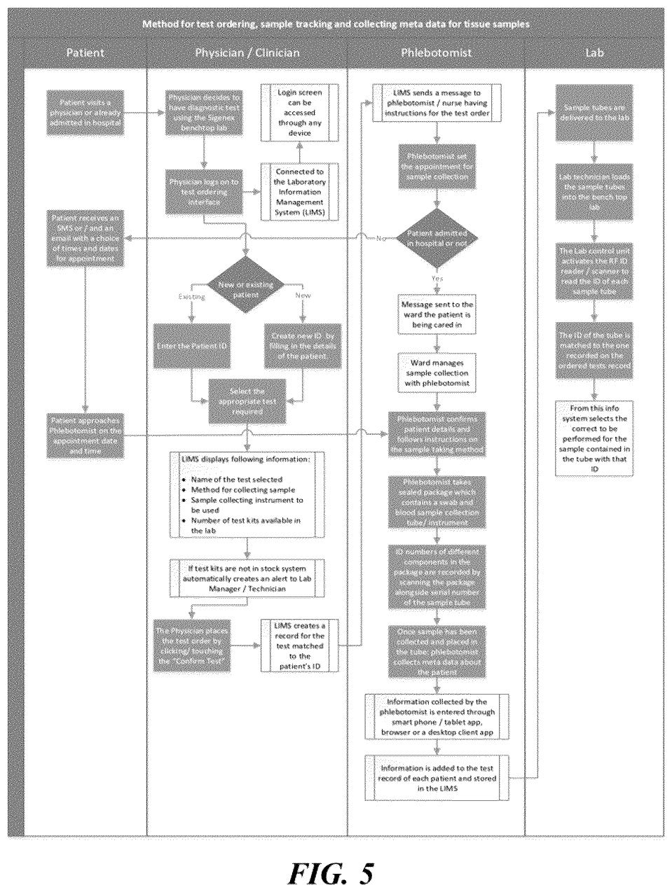

FIG. 4 schematically illustrates flow of information, process control data, and sample flow from the various applications, bench top system, and control center. The test ordering, tracking and results access application, sample scheduling application, lab manager control application, and cloud based central control center may provide information to and receive information from the LIMS. The in system analytics may provide information to the LIMS. The LIMS may provide process control data to the control unit. The control unit may provide process control data to the electromechanical controllers, actuators, process unit, sequencer, or any combination thereof. The sample may be input into the system and undergo lysing and extraction in a first chip. The extract may be provided to a second chip for sample library construction. The library may be provided to the sequencer. The results from the sequencer may be provided to an incorporated dry lab or relayed to an external dry lab. FIG. 5 schematically illustrates a process flow for sample ordering and tracking. The patient (e.g., subject), physician or clinician, phlebotomist, and lab may have access to select data related to the system and patient health as per local regulations. The patient visiting the physician or clinician may trigger a cascade of events leading to sample collection, sample processing, sample analysis, results analysis, and physician recommendations.

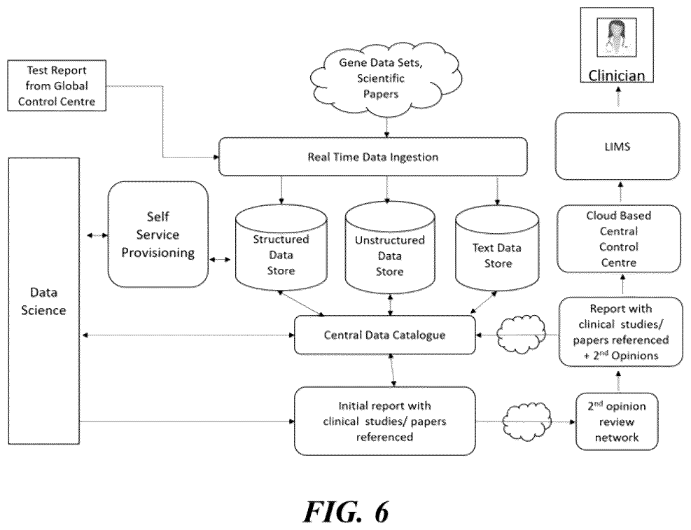

FIG. 6 schematically illustrates the data base (e.g., knowledge base) and bioinformatics system, which may be coupled to the analysis system and user devices. The knowledge base may input data in real time from gene data sets, scientific papers, and test reports from the global control center. The real time data may be structured or unstructured and may be stored in a central data catalog. The knowledge base and bioinformatics may use the data generated from the system in combination with the data from the central data catalog to generate an initial report. The initial report may include clinical studies and/or papers referenced. The report may be provided to a second opinion network for review and second opinions. The report and second opinion may be provided to the clinician through the LIMS.

The system may be configured as or may be a closed system such that the system may complete analysis of a sample with little to no operator intervention. Alternatively, or in addition to, the system may be an open system that uses operator intervention (e.g., sample or reagent handling, transferring sample between elements of the system, etc.). The system may use one time use or minimally reusable chips and minimal operator manipulation to reduce potential contamination and increase repeatability and reproducibility across devices, labs, and operators. A closed system may be configured to accept or may accept one or more consumables (e.g., sample processing chips, cartridges, reagents, buffers, etc.) prior to operation, during operation, or both prior to and during operation. The closed system may be self-contained such that, once samples and consumables are provided to the system, the system processes and analyzes the sample(s) with little to no operator intervention.

The system may include at least one lab control unit. The lab control unit may control the system modules. For example, the lab control unit may control the input module, processing module (e.g., microfluidic drive), detection module, thermal module, sequencer, dry lab, or any combination thereof. The lab control unit may be in communication with one or more external user devices, the analysis system, a global control center, or any combination thereof. The lab control unit may be integrated with the analysis system. Alternatively, the lab control unit may be separate from the analysis system.

The system may include one or more input modules (e.g., loading bays, storage bays, autosampler, etc.). The input module may be a loading bay configured to accept or may accept the sample (e.g., in a chip or in fluid form), cartridges, chips, reagents, buffers, or any combination thereof. The loading bay may transfer the sample, cartridges, chips, reagents, buffers, or any combination thereof from an operator to the system. Alternatively, or in addition to, the loading bay may be configured to accept the sample or consumable (e.g., cartridge, chip, reagents, buffer, disposables, etc.) and store the sample or consumable until requested for by the system. The reagents, buffers, etc. may be provided in cartridges and the cartridges may be provided to and input into the system. Alternatively, or in addition to, the reagents and buffers may be input into a reservoir within the system.

The input module may be a storage bay. The sample and cartridges may be stored in the system (e.g., in the loading bay or storage bay) at ambient temperatures. Alternatively, or in addition to, the samples and other materials may be stored within the system (e.g., in the loading bay or storage bay) under refrigeration. The system may or may not have a refrigeration module. In an example, the system has a refrigeration module. The refrigeration module may be in thermal communication with the input module (e.g., loading bay, storage bay, autosampler, etc.). Alternatively, or in addition to, the refrigeration module may be a part of the loading bay, storage bay, or autosampler. In an example, the refrigeration module is a part of the storage bay. The refrigeration module may permit the sample and consumables to be stored at a temperature of less than or equal to about 20 degrees Celsius (° C.), 15° C., 10° C., 5° C., 0° C., −5° C., −10° C., −20° C., or less. In an example, the refrigeration module cools the sample, consumables, or both to a temperature below ambient (e.g., about 20° C.). In another example, the refrigeration module cools the sample, consumables, or both to a temperature of less than or equal to about 4° C. In another example, the refrigeration module cools the sample, consumables, or both to less than or equal to about −20° C. Alternatively, or in addition to, the sample or consumables stored at a temperature of less than ambient may be stored external to the system and an operator load the refrigerated sample, consumables, or both at the time of the run.

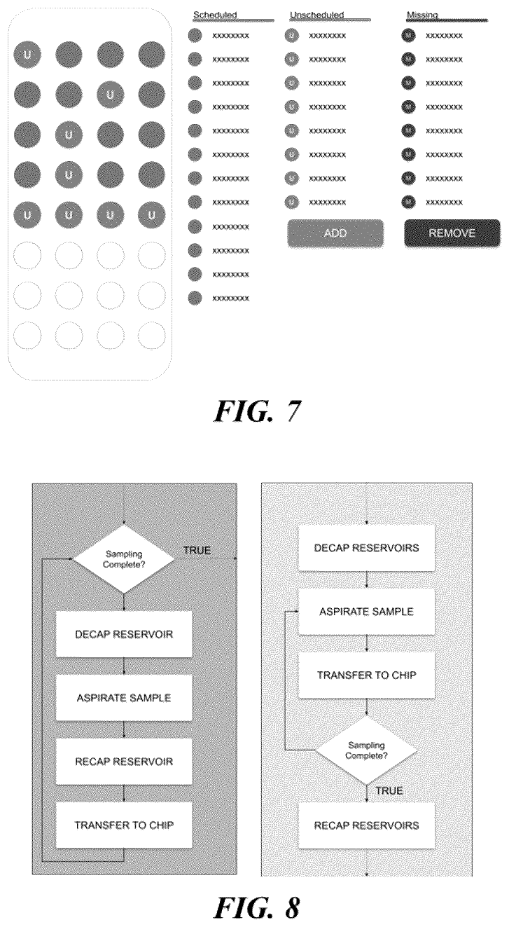

The system may or may not include an autosampler. In an example, the system includes an autosampler. The autosampler may include a loading bay and the sample, consumables, disposables, or any combination thereof may be provided to the loading bay. For example, samples and disposable pipette tips or aspiration needles may be placed in the loading bay of the autosampler and de-capping, re-capping, and transfer of samples, reagents, or both may be performed robotically. The sample reservoirs may include machine readable identifiers for identification and tracking. An operator may load samples, consumables, disposables, or any combination thereof into the system (e.g., via the autosampler) and may manually initiate a run. The run may be scheduled or unscheduled. Alternatively, or in addition to, the system may alert the operator to load the sample, consumables, disposables, or any combination thereof into the system and the run may automatically initiate. The robot may scan the loaded samples. For a scheduled run, the system may request a manifest from the laboratory management system (LIMS). For an unscheduled run, the system may request a manifest be created from the loaded samples. Control and consumables used for the run may be checked and verified against the manifest and the operator may be prompted to load any missing or incorrect items (e.g., consumables, disposables, etc.). FIG. 7 shows an example manifest of scheduled runs, unscheduled runs, and missing items. FIG. 8 shows example process flows for the autosampler. In the example, the autosampler may decap a reservoir (e.g., tube), aspirate the sample, recap the reservoir, and transfer the sample to the chip to complete sampling. Alternatively, the autosampler may aspirate the sample, transfer the sample to the chip, check for sampling to be complete, and recap the reservoir.

The autosampler may include a pipetting head or a dispensing head for needle based aspiration and dispensing. The pipetting head may couple to a pipette tip. The pipetting head may be compatible with one or more of a 5 milliliter (mL), 1 mL, 200 microliter (4), 20 μL, or 10 μL tips. The autosampler may have more than one pipetting head, each compatible with one or more pipette tip sizes. The pipetting head may have a liquid level sensor configured to or that evaluates a level of the sample to provide positioning of the pipette tip for aspiration. This may enable the pipette head to position the pipette such that the sample is aspirated fully (e.g., without entry of air into the tip). The pipette head may also provide pressure or capacitive based sensing, ultrasonic based sensing, optical based sensing, or a combination thereof for the detection of bubbles or detritus such as blood clots within the tip.

Alternatively, or in addition to, the autosampler may be configured to pierce a reservoir cap rather than decapping and pipetting the sample. The reservoirs may include self-sealing stoppers similar to evacuated blood collection devices. Sampling may include surface decontamination followed by piercing of the cap with a needle. Using cap piercing may permit the use of reservoirs with different cap types such that the system is capable of processing tubes, microwells plates, sample cups with injection ports, or any combination thereof. The autosampler may include a capacitive sensor, ultrasonic sensor, optical sensor, or any combination thereof for positioning the tip (e.g., pipette tip or needle tip) for sampling.

Alternatively, or in addition to, the sample may be provided to the system in a chip the extraction chip and the extraction chip may be loaded directly into the system (e.g., without the use of an autosampler). The chip may be transferred from the input module (e.g., loading bay) to the processing module. The processing module may interface the chip with a cartridge.

To process a sample, the sample may be provided to the input module. The input unit (e.g., autosampler) may signal the processing unit (e.g., microfluidic drive) that a sample is ready. The processing unit may rotate the cartridge in position to accept the sample. Upon loading the sample into a chip, the chip is actuated through the system according to the extraction protocol. Upon extraction, the output may be quality controlled and, upon passing, the sample may be transferred to a library preparation chip. Failures may be handled according to the assay protocol. Library preparation may be processed once the extracted output is loaded into a library preparation chip. Libraries that pass quality control may be multiplexed and transferred to the sequencer.

The sample may be analyzed using electrophoretic based quantification, fluorescence based quantification, absorption spectrophotometry, or any combination thereof. Analysis of the sample may be nucleic acid and library kit independent. As such, the chips for analysis and extraction and preparation may be on different cartridges. The cartridges may be on the same holder or may be on different holders. For example, one holder may comprise multiple cartridges with chips used for sample extraction and preparation and another holder may comprise multiple cartridges with chips for analysis.

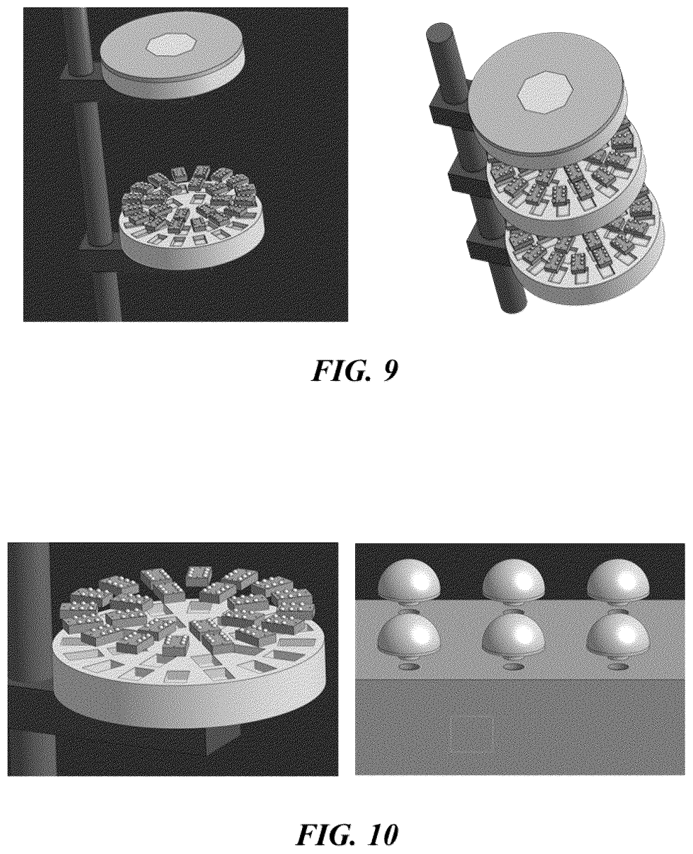

The system may include a processing module (e.g., microfluidic drive). The processing module may include a liquid handling unit, a pneumatics unit, a temperature control unit, a transfer unit, holders, actuators, sensors, or any combination thereof. The processing module may include control and logic circuitry, which may reduce the machine-to-machine interfacing to high level commands. The processing module (e.g., microfluidic drive) may be configured to interface with one or more cartridges. The processing module may comprise a standardized machine-to-holder interface for identification, loading, unloading, translation, pneumatics, fluidic and electronic communication for interfacing with the cartridges. The processing module may be configured to accept and hold one or more cartridges, chips, or both cartridges and chips. The processing module may include one or more holders that hold or are configured to hold one or more cartridges. The processing module may include at least 1, 2, 3, 4, 5, 6, 8, 10, or more holders, each configured to hold at least one cartridge. FIG. 9 shows example holders with multiple cartridges. A holder may include more than or equal to 1, 2, 3, 4, 5, 6, 8, 10, 12, 15, 20, 30, 40, 50, or more cartridges.

The cartridges may include bays configured to hold a single microfluidic chip or multiple microfluidic chips. A cartridge may include more than or equal to 1, 2, 3, 4, 5, 6, 8, 10, 12, 15, 20, 30, 40, 50, or more chips. In an example, the cartridge includes one or more chips. The number of chips may be altered or modified based on the application or chip variant. The cartridge may include holder-to-chip connectors and contact points for localized actuation or measurement (e.g., thermal cycling, magnetic bead separation, etc.). The processing module may transfer chips to and from bays using a pick and place arm. FIG. 10 shows an example cartridge and removable chips. Quality control and analyte detection may be performed by fluorometry, electrophoresis, absorption spectrophotometry, or any combination thereof. The processing module may rotate the cartridge to position a given chip an arm and the arm may position sensors or other components (e.g., pick and place components) at a given point along the axis of the arm.

The cartridge may be any shape. The cartridge may be square, rectangle, circular, disk shaped, or any other shape. In an example, the cartridge is circular or disk shaped. The extraction and preparation chips may be provided on a single disk. The disk may be on a separate holder from the holder holding the cartridges for detection. The cartridge and chips for extraction and preparation may be centrifugally driven, which may reduce the complexity of the processing unit (e.g., microfluidic drive). In an example, the cartridge may have a single large extraction and preparation chip or multiple larger surface area chips. The larger surface area of the single chip may have fewer macro interfaces, which may permit a higher density of fluidic circuits on the chip and permit a chip to last for multiple runs. Similarly, higher volumes may be achieved by stacking cartridges, for example see FIG. 10, or stacking holders and sharing arms. The combination of a variety of assay chips and sensor arms may permit different colorimetric, fluorometric, and imaging cytometric assays.

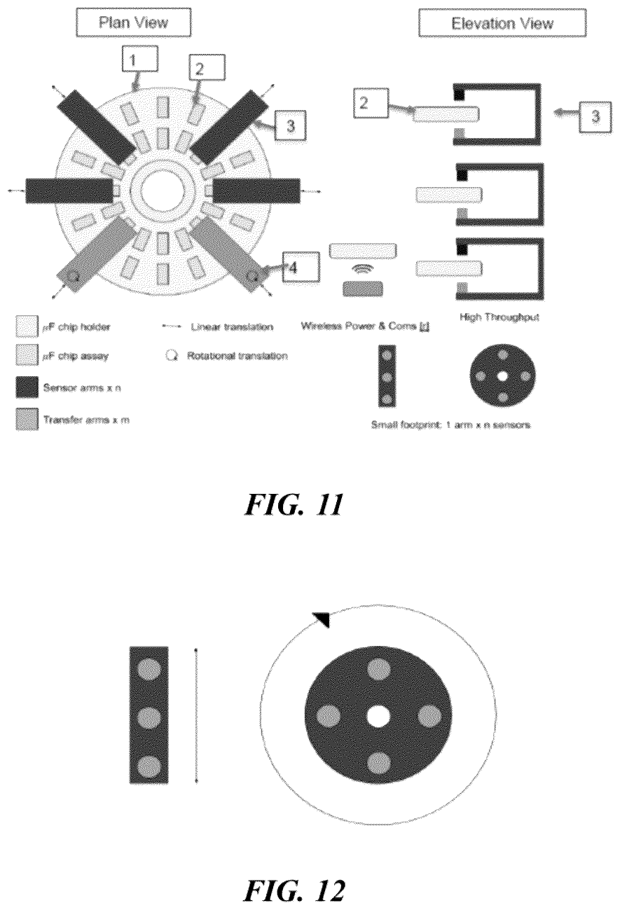

The processing module (e.g., microfluidic drive) may have one or more arms configured to position sensors, transducers, actuators, or any combination thereof for imaging cytometry, fluorometry, spectrophotometry, quantitative polymerase chain reaction (qPCR), digital polymerase chain reaction (dPCR), colorimetry, transferring chips on or off a cartridge or between bays on the same cartridge, transferring fluids to and from off-cartridge reservoirs or between chips on the cartridge, or any combination thereof. FIG. 11 schematically illustrates an example processing unit comprising multiple arms in communication with a cartridge. The cartridge may include multiple chips, as shown in the plane view of FIG. 11. Each arm may include control and logic circuitry. Alternatively, or in addition to, control and logic circuitry may be shared by multiple arms. The processing module may interface with each arm and provide signals to each arm to direct actuation, sensing, and other actions. The arm or plurality of arms may be configured for rotational translation, linear translation, or both rotational and linear translation to permit positioning of the arm relative to the cartridge and chips. Alternatively, the processing module may translate the cartridge to position a chip relative to an arm. An arm may include one or more sensors. An arm may include at least 1, 2, 3, 4, 6, 8, 10, 12, 15, 20, 30, 40, 50 or more sensors. In an example, the arm includes one or more sensors. The number of sensors per arm may be altered or modified based on the application. The sensors may be configured to detect a location of chip, temperature of a portion of the chip, detection of an analyte, etc. An arm may be configured to or may translate the sensors relative to the cartridge. FIG. 12 schematically illustrates example translation of the sensors by an arm. The processing module may have a single type of arm or may have multiple types of arms. For example, the processing module may include arms for transferring of chips and cartridges, fluidic processing, sensing, or any combination thereof. In an example, the processing module may include multiple arms stacked to permit high throughput sampling, see the elevation view of FIG. 11. The processing module may rotate a cartridge to position a given chip under or near a given arm. The arm may include electronics for driving the cartridge, digital microfluidics components, and machine-to-machine interfacing for integration into a point-of-care system or robotic platform.

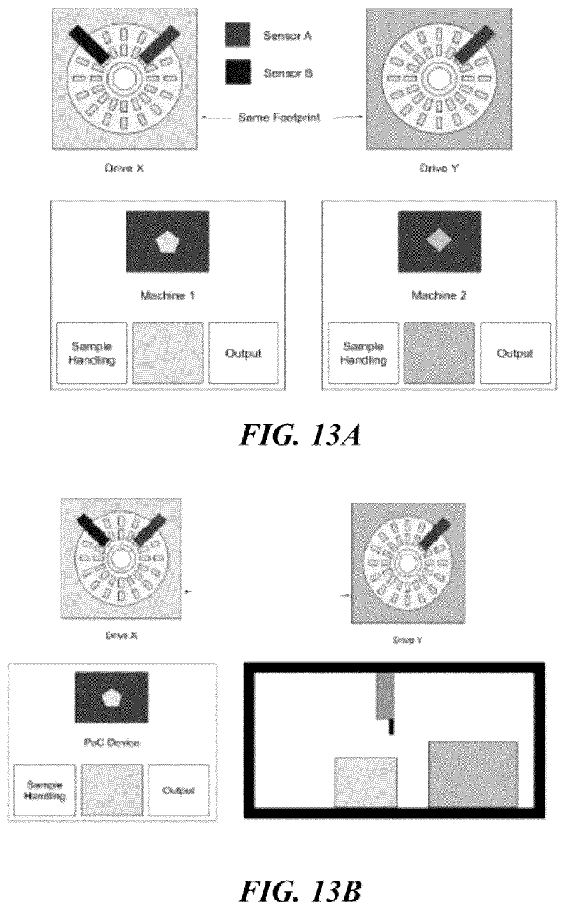

For example, the system may include a single processing module or may include multiple processing modules to permit multiple types of sample processing and analysis by exchanging the processing modules (e.g., microfluidic drive). The system may include at least 1, 2, 3, 4, 5, 6, 7, 8, 9, 10, 15, 20, or more processing modules. In an example, the system in includes one or more processing modules. The number of processing modules may be altered or modified based on the application. Alternatively, or in addition to, a processing module may be configured to permit multiple types of sample processing and analysis, for example, by providing different cartridges, chips, or using different robotic arms. FIG. 13A shows an example system with separate analysis machines, each with a single processing module. Each analysis machine may be configured to run a given set of assays due to the configuration of each machine. Therefore, assays to be run on Machine 1 may not be able to be run on machine 2. FIG. 13B shows a system with multiple processing modules integrated into the system. A system configured with multiple processing modules (e.g., microfluidic drives) may be configured to automate a wider variety of assays.

The cartridge may comprise a single material or may comprise multiple materials. The cartridge may be a multi material assembly with multiple subassemblies. The multi material assemble, the subassemblies, or both may comprise, but are not limited to, one or more metals such as stainless steel (e.g., society of automotive engineers (SAE) 316, 316L, etc.), aluminum (e.g., SAE 3000 series aluminum, 6000 series aluminum, etc.), gold, silver, platinum, copper, brass, nickel, or any combination thereof. The multi material assemble, the subassemblies, or both may comprise, but are not limited to, glass (e.g., borosilicate or quartz), plastics (e.g., composite materials, aluminum materials, glass epoxies, styrene based plastics, acrylate based plastics, methacrylate based plastics, vinyl based plastics, polyethylene based plastics, polyimide based plastics, fluoroethylene based plastics, ethylene based plastics, propylene based plastics, etc.) etc.), ceramics (e.g., oxides, carbides, nitrides, sialons, metal ceramic composites, perovskites, etc.), or any combination thereof. Cartridges may be manufactured by, but not limited to, casting, injection molding, machining, 3-dimensional printing, vacuum forming, lithography, hot embossing, wet etching, sintering, extruding, chemical bonding, thermal bonding, plasma bonding, or any combination thereof. Cartridges may be treated with various protective coatings, functionalized coatings, or both. Coatings may be applied to the cartridges by electro and electroless plating, spin coating, dip coating, spray coating, chemical vapor deposition, plasma enhanced chemical vapor deposition, sputtering annealing, calcination, pyrolysis, baking curing, powder coating, painting, other surface treatment processes, or any combination thereof. Cartridges may include assemblies and subassemblies. Sub assembly components may be formed using roll to roll coating processes. Cartridges may include electrodes configured to couple to the system, the chips, or both the system and the chips. Electrodes may include, but are not limited to, carbon electrodes, graphite electrodes, graphene electrodes, or any combination thereof.

The cartridge may comprise mesofluidic circuitry, microfluidic circuitry, or a combination of mesofluidic and microfluidic circuitry. In an example, the cartridge comprises mesofluidic circuitry. The cartridge may include pneumatics, transducers, actuators, sensors, micropumps, pressure generators, regulators, solenoid valves, electromagnets, temperature sensors, energy storage units, electronic circuitry, memory, or any combination thereof. In an example, the cartridge may have pneumatic components for the generation of pressure differentials. Alternatively, or in addition to, the pneumatic components for generation of pressure differentials may be off of the cartridge. The cartridge may include on-cartridge pressure reservoirs configured to be charged by the processing module. The pressure reservoirs may serve as a pressure source for the cartridge. Additionally, the pressure reservoirs may be filled when the cartridge is disposed in (e.g., positioned) a recharge position. The batteries or power storage units may be recharged either via wired (e.g., via brush contacts) or wirelessly when the cartridge is in the recharge position or elsewhere. Decoupling the on chip reservoirs from the pressure generation system (e.g., pneumatics module) may reduce the complexity, number of valves, and capacity used for processing multiple chips with a single pressure source.

The cartridge may comprise macro-sized contacts in fluid communication with the circuitry (e.g., mesofluidic circuitry). The chip may have microfluidic circuitry with micro-sized contacts in fluid communication with the microfluidic circuitry. Both the cartridge and the chips may have a pattern of contacts. A chip and the corresponding bay may have greater than or equal to about 1, 2, 3, 4, 5, 6, 7, 8, 10, 12, 13, 20, or more contacts. In an example, the chip and corresponding bay have one or more contacts. The number of bays may be altered or modified based on application. The pattern of contacts on the cartridge may correspond to the pattern of contacts on the chip. The contacts may provide an interface between the cartridge and the chip. The contacts may interface or provide power, electronic communication, pneumatic communication, electromagnetic communication, or any combination thereof. The cartridge may comprise a memory. The memory may store an interface protocol, driver for interfacing the chips, or both. The cartridge may include a standard interface (e.g., pattern of contacts) such that the cartridge interfaces with a variety of chips such that the cartridge is capable of performing more than one type of sample processing.

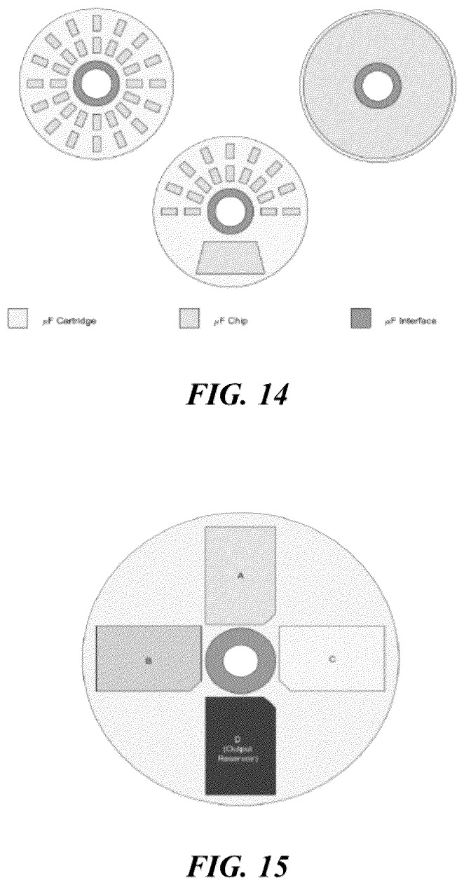

A cartridge may have one or more bays. In an example, a cartridge has a plurality of bays. The bays may be configured such that a chip removably fits within the bay. A bay may be configured such that a single type of chip may be removably coupled to that bay. Alternatively, or in addition to, the bay may be configured such that multiple types of chips may be removably coupled to the bay. A cartridge may have a single type of bay or multiple types of bays. Each type of bay may have a select geometry and/or a select pattern of contacts. Each bay may be configured to removably accept a single type of chip with a given geometry or pattern of contacts or multiple types of chips with different geometries or a pattern of contacts. FIG. 14 shows an example disk shaped cartridge with a plurality of similar chips, a cartridge with more than one chip type, and a cartridge with a single chip. The interface between the multiple chip types and the cartridge may be the same or the interface may be chip specific. Each cartridge type may be a unique combination of bays and onboard components. For example, a cartridge configured for sample extraction and purification may be different from a cartridge configured for library preparation.

FIG. 15 shows an example cartridge with four chip bays loaded with four different chip types, A, B, C, and D. Chips A, B, and C may be used to extract DNA from n samples each and prepare different libraries for NGS. Chips A, B, and C may be processed according to their assay developer protocols and the output from each chip may be transferred to D, the reservoir chip. The reservoir chip D may be transferred off-cartridge for downstream processing. A specific cartridge may be developed with a bay to hold a particular sequencer's reagent cartridge in which the prepped output may be directly introduced and then transferred off-cartridge to be placed into the sequencer with no manual intervention or intermediate handling. This standardization may permit different cartridges, and therefore, different assays to be processed by the same system.

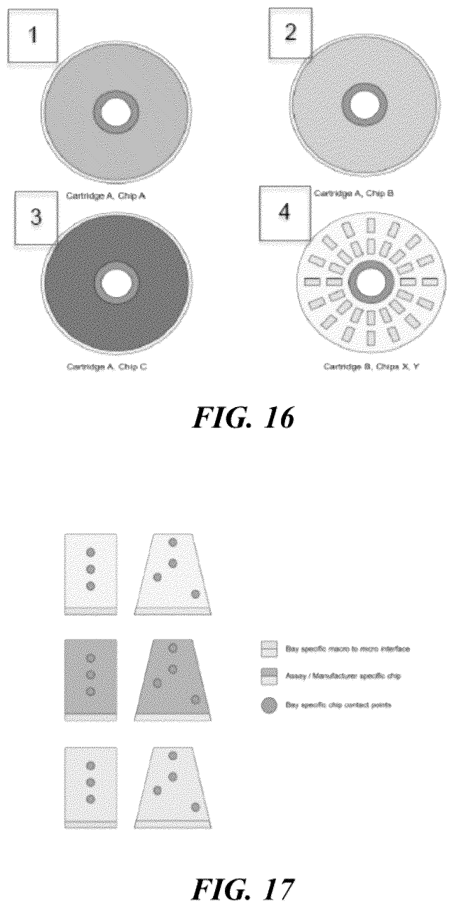

Fixed or standardized macro to micro interfaces for a given bay type may allow different chips to be processed in the same bay. For example, as shown in FIG. 16, one type of cartridge may include a single bay and another type of cartridge may have multiple bays (e.g., 32 bays) of the same type. Chips A, B, and C may be different chips, all compatible with the first type of cartridge (e.g., with the single bay) and, therefore, any one of A, B, or C may be loaded into the cartridge of the first type. In an example, chip A may be a circulating free deoxyribonucleic acid (cfDNA) extraction chip array for a number of samples, chip B may be a DNA extraction chip array for a number of samples, and chip C may be a genomic DNA (gDNA) and library preparation chip for a number of samples. Each of chips A, B, and C may comprise components (e.g., reagents, buffers, assay components, etc.) from the same manufacturer or may comprise components from different manufacturers. The chips may be processed in the same cartridge as long as they are compatible with the cartridge bay. When chip A is loaded into the cartridge, as chip A's protocol does not include incubation at an elevated temperature, the chip does not include thermal pads and the thermal transducer on the bay may not be actuated when chip A is processed. However, when chip B or C is loaded into the bay, actuation may occur according to each chip's protocol. The three chip types, A, B, and C may use magnetic bead separation and, as such, the three chips may have the corresponding separation chambers aligned over the electromagnetic contact points of the bay. This standardization of interfaces and protocols may allow assay designers to easily integrate their assays into cartridges and chips for automation and processing by the system. As such, an ecosystem of assay developers may permit a wide range of application and highly effective assays continuously being deployed which may otherwise be challenging for a single entity to develop.

The cartridge may have at least 1, 2, 3, 4, 8, 12, 16, 24, 32, 64, or more bays. In an example, the cartridge has one or more bays. A bay may be configured to hold a single chip or may be configured to removably hold multiple chips. A bay may be configured to hold a single type of chip or may be configured to hold multiple types of chips. Each bay may have a fixed geometry, contact topology and macro to micro interface for pneumatics, fluidics, electronics, contact based transducers, probes, actuators, or any combination thereof. Two bays with the same geometry but different contact topology (e.g., pattern of contacts) or macro to micro interfaces may be considered different by types. The contact points may provide power, communication, transducers (e.g., electromagnets for magnetic bead separation) which may be used to process a chip in a bay parallel with other bays. Sequential contact points may be accessed through off-cartridge robotic arms and may be located anywhere on the chip. FIG. 17 schematically illustrates examples of bay geometries and contact point topologies.

The processing module may be configured to transfer or may transfer the sample from one chip to another chip. Alternatively, or in addition to, the processing module may transfer the sample from one location in a chip to another location in the same chip. The processing module may be configured to transfer or may transfer a chip from one cartridge to another cartridge.

The chips may be formed out of or may comprise glass (e.g., borosilicate, quartz, etc.), plastic (e.g., composite materials, aluminum materials, glass epoxies, styrene based plastics, acrylate based plastics, methacrylate based plastics, vinyl based plastics, polyethylene based plastics, polyimide based plastics, fluoroethylene based plastics, ethylene based plastics, propylene based plastics, etc.), ceramics (e.g., oxides, carbides, nitrides, sialons, metal ceramic composites, perovskites, etc.), printed circuit boards, or any combination thereof. The chip may be manufactured by lithographic processes, injection molding, machining, 3-dimensional printing, vacuum forming, lithography, hot embossing, wet etching, sintering, extruding, chemical, thermal or plasma bonding, or any combination thereof. The chip may be treated with various protective coatings, functionalized coatings, or both. Coatings may be applied via electro and electroless plating, spin coating, dip coating, spray coating, chemical vapor deposition, plasma enhanced chemical vapor deposition, sputtering, calcination, pyrolysis, baking, curing, powder coating, painting, other surface treatment processes, or any combination thereof. Chips may include assemblies and subassemblies. Sub assembly components may be formed using roll to roll coating processes. Chips may include electrodes configured to electrically couple the chips to the cartridges. Electrodes may include, but are not limited to, carbon electrodes, graphite electrodes, graphene electrodes, or any combination thereof.

The chips may include reagents for a given assay. Some reagents may be stored under refrigeration conditions. Chips may be pre-mounted with the assay components. Alternatively, or in addition to, the assay components may be stored off of the chip and added to the chip during sample processing, as shown in FIG. 10. A chip may be compatible with multiple reagent kits such that performing a different assay may include substituting one reagent kit with another.

A chip may be transferable from one cartridge to another cartridge. Alternatively, or in addition to, a chip may be transferable from one location on a cartridge to another location on the same cartridge. The cartridge may be configured to process and analyze a sample via directing the sample from one chip (e.g., nucleic acid extraction chip) to another chip (e.g., library preparation chip). Alternatively, or in addition to, a chip with the sample may be transferred from one cartridge to another cartridge for further processing or analysis.

The chips may be configured to or otherwise process the sample. The chip may be configured to or may perform at least one function during processing of the sample. The functions performed by the chip may include extracting nucleic acid from the sample, library preparation, amplifying the sample, sequencing the sample, separating or otherwise purifying components of the sample, assaying the sample, or any combination thereof. Chips may perform a single function (e.g., extraction, library preparation, etc.) or a chip may perform multiple functions. A chip may perform one or more functions. A chip may perform at least 1, 2, 3, 4, 5, 10, 15, 20, or more functions. In an example, the chip performs one or more functions. The number of functions a chip performs may be based on the application of the chip. For example, one chip may perform nucleic acid extraction of the sample and another chip may perform library preparation. Chips that perform fewer functions (e.g., one or two functions) may have simpler circuits for each process, thereby reducing manufacturing complexities and lowering consumable costs when upstream processes fail. Additionally, modification of a single circuit (e.g., circuit for sample extraction) may not have an impact on the design of another circuit (e.g., library preparation circuit). For example, in the event that an extracted nucleic acid output is unviable for sequencing, library preparation is not performed, which may save on reagents, consumables, sequencing reads, and computing resources.

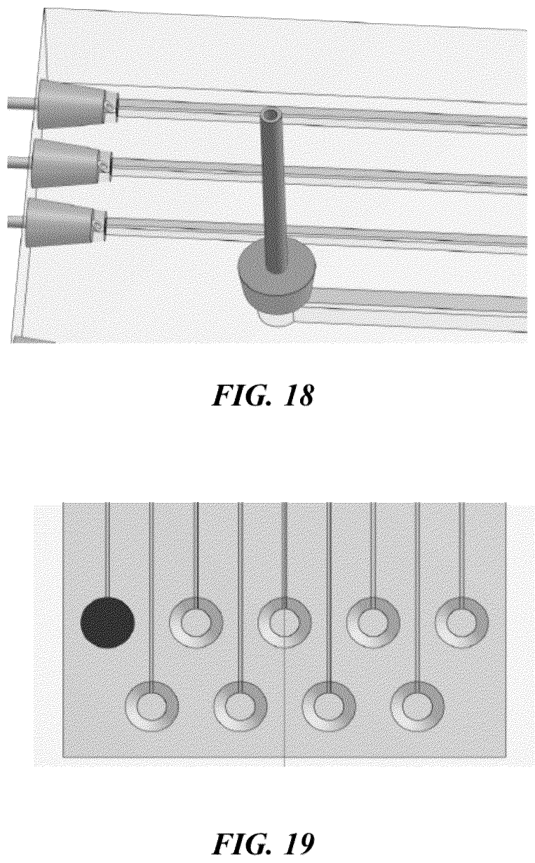

The chips may include a port or may include multiple (e.g., a plurality) of ports. A chip may include greater than or equal to 1, 2, 4, 6, 8, 10, 12, 15, 20, 25, 30, 40, 50, or more ports. In an example, the chip includes one or more ports. The ports may be sealed with a self-sealing material similar to evacuated blood collection tubes or injection ports. The seal may be bonded in place. The port may be drafted to reduce dead volume. Fluid may be introduced or extracted from a port via a needle puncturing the seal of the port. FIG. 18 shows an example of a connector puncturing the self-sealing material of the chip port. The self-sealing material may be embedded into the hole of the port and the needle may pierce the self-sealing material such that the interface takes up no more surface area of the chip than the size of the port hole. The small interface size may permit multiple ports to be placed at a close pitch, reducing dead space, thus allowing for denser circuits and leak free multiport connections, as shown in FIG. 19. The ports may be pneumatically sealed. Pneumatic sealing may reduce the risk of contamination of the chip by environmental contamination. Pneumatically sealed ports may permit the use of rechargeable on chip pressure reservoirs without dedicated pneumatic connections. The self-sealing material may also simplify decontamination. For example, decontamination of the chip may be performed by swabbing the surface of the self-sealing material with a suitable decontaminant (e.g., bleach, alcohol, etc.), spot irradiating the surface with an appropriate waveband (e.g., UVB or UVC), or both swabbing and spot irradiating.

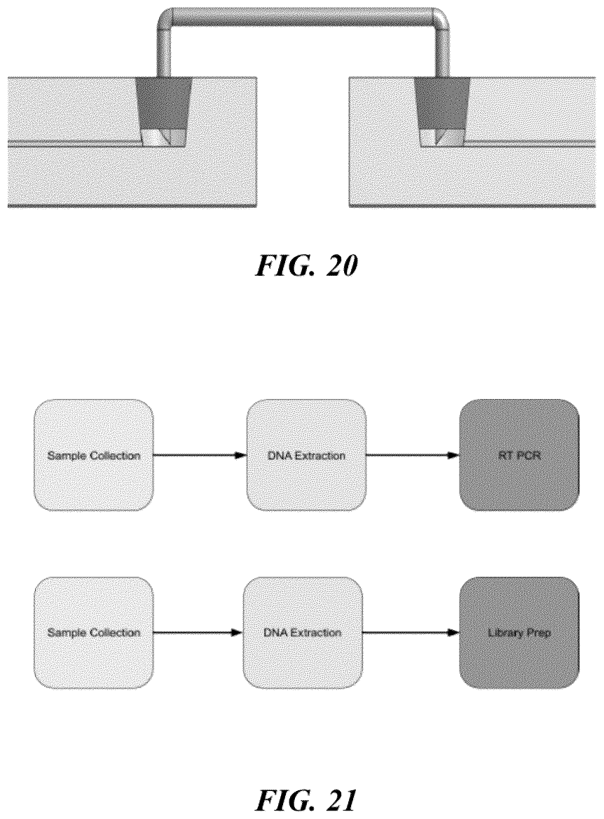

The chips may be configured to be or may be fluidically connected using disposable connectors. Alternatively, or in addition to, the connector may not be disposable. The connector may be formed from compatible stainless steel alloys (e.g., SAE 316, 316L, etc.), polytetrafluoroethylene (PTFE), polyoxymethylene (POM), other non-reactive biocompatible materials, or any combination thereof. In an example, the connector is formed of PTFE and is disposable. In another example, the connector is formed of stainless steel and is disposable. The connectors may be configured with needle-like projections that puncture the self-sealing material. The connectors may or may not be needles. For example, the connector may be a double sided needle, single sided needle, or other needle configuration. The needle may be a sterilized, medical grade needle. Alternatively, the connector may not be sterilized and may not be medical grade. Alternatively, the connector may not include a needle. FIG. 20 schematically illustrates a disposable, double sided needle connector. The connectors may provide fluidic connection between chips, between a chip and the cartridge, or between cartridges. In an example, the connector provides a fluidic connection between chips. In another example, the connector provides fluidic connection between a chip and the cartridge. The chips or chips and cartridge may be interconnected manually (e.g., by an operator) or by robotic automation. The use of robotic operation for connecting chips and chips to cartridges may permit modularization of the microfluidic components and may reduce potential contamination and variation during processing. Modularization may also simplify manufacturing and the supply chain. Furthermore, modularization of the chips and chip to chip interfaces may simplify surface mounted components and probe introduction.

The use of connectors between chips may permit samples to be loaded directly onto a chip (e.g., the collection device may include a chip) and input into the system to reduce potential contamination risks and operator error. The samples may or may not be collected through a winged needle infusion. The sample collection chip may be configured to or may separate plasma and serum upon collection. Alternatively, the sample collection chip may store whole blood. The sample chip may or may not include microfluidic circuitry for sample separation, extraction, purification, or any combination thereof. Separating sample extraction, purification, and other preparation processes between chips may reduce waste and cost and increase system efficiency. For example, as shown in FIG. 21, a reverse transcription polymerase chain reaction (RT PCR) assay and NGS assay both use nucleic acid extraction prior to polymerase chain reaction (PCR) and library preparation, respectively. If the chips are integrated such that the sample preparation and assay are performed in a single chip, in the event that the extraction quantity or quality does not meet the standard for the assay, the entire chip may be disposed of, including expensive reagents for downstream processing. By separating the processes between multiple chips, failure of one part of the process may prevent extraneous waste of materials and resources.

The system may include a thermal module. The thermal module may be configured to contact at least a portion of a cartridge, a chip, or both a cartridge and a chip. The thermal module may be configured to control a temperature of a portion or all of the cartridge, chip, or both. The thermal module may provide a temperature to the cartridge or chip of greater than or equal to about −30° C., −20° C., −10° C., 0° C., 10° C., 20° C., 30° C., 40° C., 50° C., 60° C., 70° C., 80° C., 90° C., 100° C., 110° C., 120° C., or more. The thermal module may maintain or modulate a temperature (e.g., provide a thermal range) of the cartridge or chip from about −30° C. to −20° C., −30° C. to −10° C., −30° C. to 0° C., −30° C. to 10° C., −30° C. to 20° C., −30° C. to 30° C., −30° C. to 40° C., −30° C. to 50° C., −30° C. to 60° C., −30° C. to 70° C., −30° C. to 80° C., −30° C. to 90° C., −30° C. to 100° C., −30° C. to 110° C., −30° C. to 120° C., −20° C. to −10° C., −20° C. to 0° C., −20° C. to 10° C., −20° C. to 20° C., −20° C. to 30° C., −20° C. to 40° C., −20° C. to 50° C., −20° C. to 60° C., −20° C. to 70° C., −20° C. to 80° C., −20° C. to 90° C., −20° C. to 100° C., −20° C. to 110° C., −20° C. to 120° C., −10° C. to 0° C., −10° C. to 10° C., −10° C. to 20° C., −10° C. to 30° C., −10° C. to 40° C., −10° C. to 50° C., −10° C. to 60° C., −10° C. to 70° C., −10° C. to 80° C., −10° C. to 90° C., −10° C. to 100° C., −10° C. to 110° C., −10° C. to 120° C., 0° C. to 10° C., 0° C. to 20° C., 0° C. to 30° C., 0° C. to 40° C., 0° C. to 50° C., 0° C. to 60° C., 0° C. to 70° C., 0° C. to 80° C., 0° C. to 90° C., 0° C. to 100° C., 0° C. to 110° C., 0° C. to 120° C., 10° C. to 20° C., 10° C. to 30° C., 10° C. to 40° C., 10° C. to 50° C., 10° C. to 60° C., 10° C. to 70° C., 10° C. to 80° C., 10° C. to 90° C., 10° C. to 100° C., 10° C. to 110° C., 10° C. to 120° C., 20° C. to 30° C., 20° C. to 40° C., 20° C. to 50° C., 20° C. to 60° C., 20° C. to 70° C., 20° C. to 80° C., 20° C. to 90° C., 20° C. to 100° C., 20° C. to 110° C., 20° C. to 120° C., 30° C. to 40° C., 30° C. to 50° C., 30° C. to 60° C., 30° C. to 70° C., 30° C. to 80° C., 30° C. to 90° C., 30° C. to 100° C., 30° C. to 110° C., 30° C. to 120° C., 40° C. to 50° C., 40° C. to 60° C., 40° C. to 70° C., 40° C. to 80° C., 40° C. to 90° C., 40° C. to 100° C., 40° C. to 110° C., 40° C. to 120° C., 50° C. to 60° C., 50° C. to 70° C., 50° C. to 80° C., 50° C. to 90° C., 50° C. to 100° C., 50° C. to 110° C., 50° C. to 120° C., 60° C. to 70° C., 60° C. to 80° C., 60° C. to 90° C., 60° C. to 100° C., 60° C. to 110° C., 60° C. to 120° C., 70° C. to 80° C., 70° C. to 90° C., 70° C. to 100° C., 70° C. to 110° C., 70° C. to 100° C., 80° C. to 90° C., 80° C. to 100° C., 80° C. to 110° C., 80° C. to 120° C., 90° C. to 100° C., 90° C. to 110° C., 90° C. to 120° C., 100° C. to 110° C., 100° C. to 120° C., or 110° C. to 120° C. In an example, the thermal range is from about 50° C. to 100° C. In another example, the thermal range is from about 55° C. to 98° C. The thermal module may be configured to provide thermal cycling of the chips. Thermal cycling may be used for nucleic acid amplification.

Thermal cycling may include cycles of incubating the chips at a temperature sufficiently high to denature nucleic acid molecules for a duration followed by incubation of the chips at an extension temperature for an extension duration. Denaturation temperatures may vary depending upon, for example, the particular nucleic acid sample, the reagents used, and the reaction conditions. In some embodiments, a denaturation temperature may be from about 80° C. to about 110° C. In some embodiments, a denaturation temperature may be from about 85° C. to about 105° C. In some embodiments, a denaturation temperature may be from about 90° C. to about 100° C. In some embodiments, a denaturation temperature may be from about 90° C. to about 98° C. In some embodiments, a denaturation temperature may be from about 92° C. to about 95° C. In some embodiments, a denaturation temperature may be at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., at least about 96° C., at least about 97° C., at least about 98° C., at least about 99° C., or at least about 100° C.

The duration for denaturation may vary depending upon, for example, the particular nucleic acid sample, the reagents used, and the desired reaction conditions. In some embodiments, the duration for denaturation may be less than about 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. In an alternative embodiment, the duration for denaturation may be no more than about 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Extension temperatures may vary depending upon, for example, the particular nucleic acid sample, the reagents used, and the desired reaction conditions. In some embodiments, an extension temperature may be from about 30° C. to about 80° C. In some embodiments, an extension temperature may be from about 35° C. to about 75° C. In some embodiments, an extension temperature may be from about 45° C. to about 65° C. In some embodiments, an extension temperature may be from about 55° C. to about 65° C. In some embodiments, an extension temperature may be from about 40° C. to about 60° C. In some embodiments, an extension temperature may be at least about 35° C., at least about 37° C., at least about 40° C., at least about 41° C., at least about 45° C., at least about 50° C., at least about 55° C., at least about 60° C., at least about 65° C., at least about 70° C., at least about 75° C., at least about 80° C., or more.

Extension time may vary depending upon, for example, the particular nucleic acid sample, the reagents used, and the desired reaction conditions. In some embodiments, the duration for extension may be less than about 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. In an alternative embodiment, the duration for denaturation may be no more than about 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

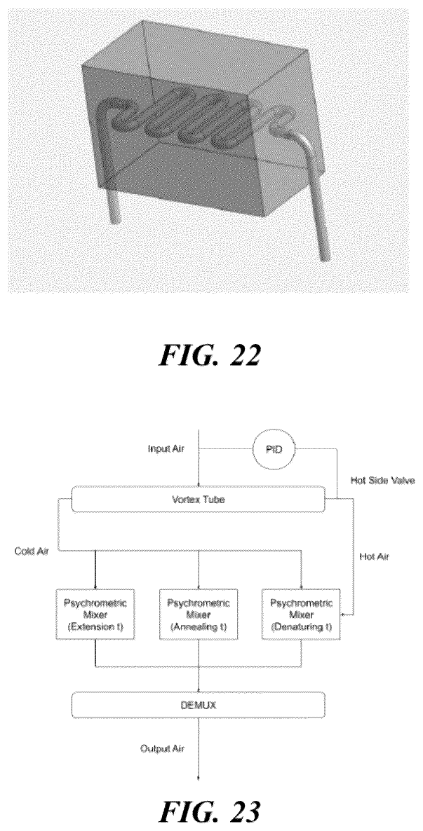

To reduce costs, the chips may be formed out of polymeric or plastic materials (e.g., composite materials, aluminum materials, glass epoxies, styrene based plastics, acrylate based plastics, methacrylate based plastics, vinyl based plastics, polyethylene based plastics, polyimide based plastics, fluoroethylene based plastics, ethylene based plastics, propylene based plastics, etc.). In an example, the chips are formed out of polymethyl methacrylate (PMMA), polystyrene (PS), polycarbonate (PC), polytetrafluoroethylene (PTFE), polyethylene terephthalate (PET), high-density polyethylene (HDPE), polyvinyl chloride (PVC), low-density polyethylene (LDPE), polypropylene (PP), or any combination thereof. However, some polymeric materials may be prone to channel deformation at the elevated temperatures. To reduce material waste and cost, a separate chip may be used for thermal cycling. Alternatively, or in addition, thermal cycling may be performed in another process chip (e.g., sample construction chip). Chips used for thermal cycling may be formed out of thermally conductive materials or may comprise regions formed of thermally conductive materials. Chips used for thermal cycling may be stacked on a cartridge or surface mounted to the cartridge or chip. In an example, thermal cycling occurs in a surface mounted chip comprising a heat exchanger, as shown in FIG. 22. The heat exchanger may be thermally insulted from the chip substrate such that the portion of the chip comprising the sample is thermal cycled and the rest of the chip may not be thermal cycled. For example, the heat exchanger may be disposed on or adjacent to a portion of the chip to localize the temperature gradient.

The chip may be a sample collection chip. The sample collection chip may accept and hold whole blood samples, whole blood samples with plasma separation, plasma samples, bronchoalveolar lavage samples, urine sample, sputum samples, synovial fluid samples, liquified stool samples, tissue fluid samples, any other biological fluid sample, or any combination thereof. The chip may be a nucleic acid extraction chip. The nucleic acid extraction chip may extract, isolate, or both extract and isolate nucleic acids (e.g., cell free DNA, circulating tumor DNA, DNA, cell free RNA, circulating tumor RNA, RNA, etc.). The chip may be a library preparation chip, quality control chip (e.g., electrophoretic, fluorometric, spectrophotometric, etc.), or any combination thereof.

Temperature control may be important for many biological and chemical reactions. For example, precise temperature control may be used for biological assays such as PCR, temperature gradient electrophoresis, and other clinical chemistries. Temperature control for microfluidic devices may be via a contact based approach or a contactless approach. Contact based thermal control may include the use of thermoelectric heating and cooling devices or resistive heating for thermal control. Contact based thermal control may not scale well to batch processing of assays. Contactless approaches may include lasers, microwaves, or a combination thereof. These types of contactless thermal control may be complex to implement and expensive to develop and use. Alternatively, the contactless thermal control may be provided by a pneumatic system. In an example, the thermal module is configured to provide contactless temperature control via a pneumatic system. The thermal module may include a Rank-Hilsch vortex tube that provides contactless temperature control. As shown in FIG. 23, a Rank-Hilsch vortex tube may separate compressed air into hot and cold streams such that the hot stream may provide heating and the cold stream may provide cooling. The Rank-Hilsch system may have no or few moving parts, use low pressures (e.g., from about 1 to 10 bar), and provide large temperature differentials (e.g., from about −50° C. and 200° C.). Speed of temperature ramp and the temperature differential may be controlled by regulating the cold fraction and input pressure into the vortex tube. Temperature precision and fast temperature ramping (e.g., optimized temperature ramping and control) may be permitted by blending the hot and cold outputs using a psychrometric process similar to heating, ventilation, and air conditioning systems. Temperature regulation may be achieved by focusing an air jet from the vortex tube onto the incubation chamber or chip. In this example, the output air may be the thermal fluid. Pneumatic temperature control may permit controlling temperatures for extension, annealing, and denaturing simultaneously (e.g., with a single source), thus permitting continuous sample processing with low energy consumption.

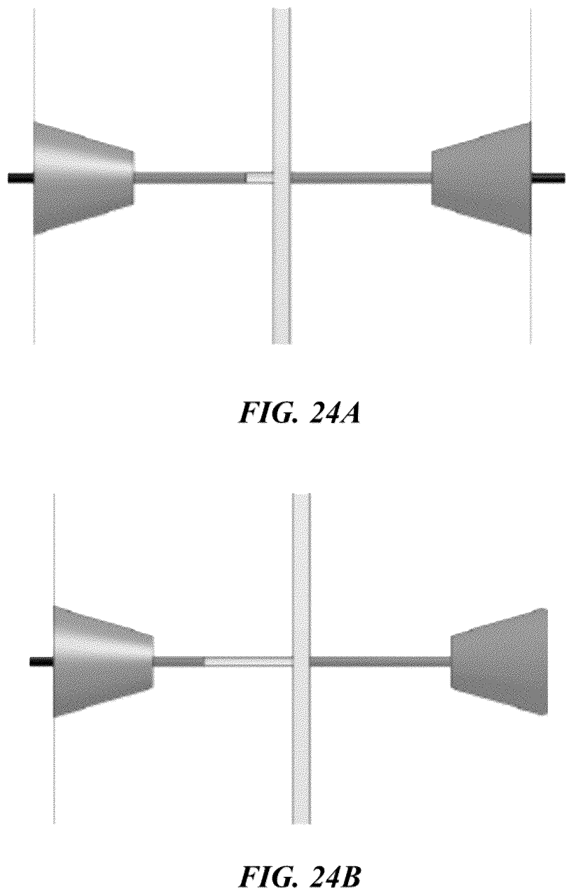

The system may comprise one or more detection modules configured to analyze the sample. The detection module may be configured to quality control a sample prior to analysis of the sample (e.g., prior to sequencing). Alternatively, or in addition to, the detection module may be used for analysis of the sample. Quality control of the sample may include determining the quantity and quality of a sample prior to analysis. The detection module may include sensors and transducers for spectroscopic analysis, fluorometric analysis, colorimetric analysis, potentiometric analysis, electrophoretic analysis, cytometric analysis, or any combination thereof. In an example, the detection module includes a spectrophotometer. The spectrophotometer may be a fixed pathlength spectrophotometer or a variable pathlength spectrophotometer. The variable pathlength spectrophotometer may have a pathlength that varies from about 0.01 millimeters (mm) to about 0.2 mm. A variable pathlength spectrophotometer may be advantageous for determine sample quantity and quality for low quantities and concentrations. Integration of absorbance measurement may be complex for in plane variable pathlength digital microfluidic circuits as these measurements may account for droplet lens effects. Alternatively, fixed pathlength spectroscopy may be prone to short pathlength sensitivity. The use of cross-channel waveguides, as shown in FIGS. 24A and 24B, may permit in-plane variable pathlength absorbance measurements. A waveguide circuit may be a cross-channel circuit. One end of the cross-channel circuit may be coupled to a light source and the other to a photodetector. A waveguide may be fixed to the light source or may be integrated into the chip itself (e.g., a fused silica waveguide). Another waveguide may be introduced into the channel. The fiberoptic coaxial to the piercing needle and of the same cross-sectional dimension as the channel. The fiberoptic may act as a hydraulic piston. The hydraulic piston may be positioned using a linear stage and reading may be taken at different positions to determine a slope of the measurement. Alternatively, the fiberoptic may be displaced hydraulically and the readings may be taken during the linear phase of the pressure curve.

The detection module may be configured to or may otherwise detect an indicator provided with the sample. The indicator may be added to the sample upon collection, during processing, or prior to analysis. The indicator may be provided as a reagent in a chip or cartridge. The indicator may be analyte specific or may be a universal indicator. The indicator may permit the detection module to analyze the sample. Analysis may include detecting the presence or absence of a biological or chemical component, detecting a biological or chemical reaction, or both. An indicator may include a molecule comprising a detectable moiety. The detectable moiety may include radioactive species, fluorescence labels, chemiluminescent labels, enzymatic labels, colorimetric labels, or any combination thereof. Non-limiting examples of radioactive species include ³H, ¹⁴C, ²²Na, ³²P, ³³P, ³⁵S, ⁴²K, ⁴⁵Ca, ⁵⁹Fe, ¹²³I, ¹²⁴I, ¹²⁵I, ¹³¹I, or ²⁰³Hg. Non-limiting examples of fluorescent labels include fluorescent protein, an optically active dye (e.g., a fluorescent dye), an organometallic fluorophore, quantum dots, or any combination thereof. Non-limiting examples of chemiluminescent labels include enzymes of the luciferase class such as Cypridina, Gaussia, Renilla, and Firefly luciferases. Non-limiting examples of enzymatic labels include horseradish peroxidase (HRP), alkaline phosphatase (AP), beta galactosidase, glucose oxidase, or other labels known in the art.

An indicator molecule may be a fluorescent molecule. Fluorescent molecules may include fluorescent proteins, fluorescent dyes, and organometallic fluorophores. In some embodiments, the indicator molecule is a protein fluorophore. Protein fluorophores may include green fluorescent proteins (GFPs, fluorescent proteins that fluoresce in the green region of the spectrum, generally emitting light having a wavelength from 500-550 nanometers), cyan-fluorescent proteins (CFPs, fluorescent proteins that fluoresce in the cyan region of the spectrum, generally emitting light having a wavelength from 450-500 nanometers), red fluorescent proteins (RFPs, fluorescent proteins that fluoresce in the red region of the spectrum, generally emitting light having a wavelength from 600-650 nanometers). Non-limiting examples of protein fluorophores include mutants and spectral variants of AcGFP, AcGFP1, AmCyan, AmCyan1, AQ143, AsRed2, Azami Green, Azurite, BFP, Cerulean, CFP, CGFP, Citrine, copGFP, CyPet, dKeima-Tandem, DsRed, dsRed-Express, DsRed-Monomer, DsRed2, dTomato, dTomato-Tandem, EBFP, EBFP2, ECFP, EGFP, Emerald, EosFP, EYFP, GFP, HcRed-Tandem, HcRed1, JRed, Katuska, Kusabira Orange, Kusabira Orange2, mApple, mBanana, mCerulean, mCFP, mCherry, mCitrine, mECFP, mEmerald, mGrape1, mGrape2, mHoneydew, Midori-Ishi Cyan, mKeima, mKO, mOrange, mOrange2, mPlum, mRaspberry, mRFP1, mRuby, mStrawberry, mTagBFP, mTangerine, mTeal, mTomato, mTurquoise, mWasabi, PhiYFP, ReAsH, Sapphire, Superfolder GFP, T-Sapphire, TagCFP, TagGFP, TagRFP, TagRFP-T, TagYFP, tdTomato, Topaz, TurboGFP, Venus, YFP, YPet, ZsGreen, and ZsYellow1.

An indicator molecule may be a fluorescent dye. Non-limiting examples of fluorescent dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

An indicator molecule may be an organometallic fluorophore. Non limiting examples of organometallic fluorophores include lanthanide ion chelates, nonlimiting examples of which include tris(dibenzoylmethane) mono(1,10-phenanthroline)europium(III), tris(dibenzoylmethane) mono(5-amino-1,10-phenanthroline)europium (III), and Lumi4-Tb cryptate.

The system may comprise assay specific modules or units. The assay specific module or unit may be integrated into the system (e.g., into the detection module). Alternatively, or in addition to, the assay specific module or unit may be separate from the detection module. Assay specific modules or units may include a sequencing unit. The sequencing unit may be configured to or may sequence at least a portion of the sample.

The system may comprise an alert module. The alert module may be configured to or may otherwise monitor the system. The alert module may alert a user (e.g., operator, technician, etc.) is the system is operating outside of set operating parameters. Alternatively, or in addition to, the alert module may alert a user if the system has been opened or otherwise tampered with.

The system may comprise an authentication module, tracking module, or both for validation, tracking, or both validation and tracking. In an example, the authentication module and tracking module are integrated together. Each cartridge or chip may have an authentication unit, such as a machine readable identifier, for validation and/or tracking of the cartridge or chip. The authentication unit may provide cryptographic security. The tracking module may be configured to or may track the sample as it is processed by the system, consumption of consumables, or both. The cartridge may include a consumable chip that tracks the consumption of consumables. The consumable chip may provide cryptographic authorization. The consumable chip may include a burnable fuse such that once a bit is set it may not be changed. The cartridges, chips, or both the cartridges and chips may include at least one machine readable identifier configured to be tracked or is tracked by the tracking module as the sample is processed by the system. In an example, the cartridge has a crypto secured identification mechanism for verification and traceability. The cartridge interfacing protocol (e.g., cartridge drivers) may be stored on cartridge or stored in the system. The machine readable identifier may be a barcode, quick response code, dot code, ultra-high frequency crypto transponder, or any combination thereof. The cartridges, chips, or both the cartridges and chips may include at least one human readable identifier that a user of the system uses to track the sample. The human readable identifier may be embedded with an RFID, printed barcode, or both RFID and printed barcode that is machine readable. In an example, the cartridges, chips, or both the cartridges and chips include at least one machine readable identifier and at least one human identifier.

### Devices and Methods for Sample Collection

In an aspect, the present disclosure provides devices for collecting a sample. The device may include an inlet port, one or more chips, and an adapter. The inlet port may be configured to or may otherwise collect a sample from a subject. The chips may be in fluid communication with the inlet port. The adapter may be in fluid communication with both the chips and the inlet port. The adapter may be configured to direct or may otherwise direct sample from one or more mesofluidic channels of the inlet port to one or more microfluidic channels of the chips.

The quality of analysis may depend on the analytical phase of laboratory testing, preanalytical phase, or both. It is estimated that preanalytical errors may account for up to 70% of total testing errors. Preanalytical errors may result in additional or unnecessary tests and increase financial burden on healthcare. Preanalytical errors may account for between 0.23% and 1.2% of total hospital operating costs and more than 25% of preanalytical errors may result in improper patient care. Therefore, improvement and standardization of preanalytical practices may decrease costs and increase patient care.

For example, phlebotomy may be one of the most widely used invasive procedures in laboratory medicine. Phlebotomy may be performed worldwide in heterogenous settings (e.g., hospitals, home-base, field-based, etc. by individuals with varying levels of training and educational backgrounds. There may also be wide variation in technique, equipment, and protocols. Phlebotomy may be the largest source of preanalytical errors in laboratory medicine. Select organizations (e.g., World Health Organization, European Federation of Clinical Chemistry and Laboratory Medicine, Clinical & Laboratory Standards Institute, etc.) may provide guidelines for phlebotomy, but there may not be harmonization between guidelines, compliance may be pore, and there may be a lack of continual education and implementation tracking. For example, and as shown in Table 1, various nations may have different pre-sampling fasting guidelines for the same laboratory test. Patient identification and testing requirements, patient position, activity, dietary and fasting status, medication, supplementation, site selection and sanitization, time of draw, order of draw, handling, storage, and transportation may play a significant role in testing accuracy.

TABLE 1
phlebotomy guidelines for various medical tests

| | Glucose | ALP | Triglycerides |
|---|---|---|---|
| USA | 8 h fast | Fasting preferred | 9-12 h fasting |
| | Recommended | but not required | recommended. No |
| | | | alcohol for 24 h prior. |
| UK | 8 h fast | Fasting preferred | 9-12 h fasting |
| | Recommended | but not required | recommended. No |
| | | | alcohol for 24 h prior. |
| Australia | 8-10 h fast | Fasting overnight | 10-16 h fasting |
| | Recommended | recommended | recommended. No |
| | | | alcohol for 24 h prior. |
| Germany | 12 h fast | Fasting overnight is | 12-14 h fasting |
| | recommended | recommended | recommended. No |
| | | | alcohol for 24 h prior. |
| Czech | 8-10 h fast | Fasting overnight | 12-14 h fasting |
| Republic | Recommended | recommended | recommended. No |
| | | | alcohol for 24 h prior. |
| Italy | 8 h fast | No requirements | Fasting |
| | Recommended | | recommended. |

For select laboratory tests, guidelines may be well established, but compliance may remain poor due to the large heterogeneity of professionals performing phlebotomy (e.g., in resource poor settings). For example, phlebotomists may have little or no formal training in sample collection and handling, may not have access to safety equipment and post exposure prophylaxis, may not have access to proper sample handling and storage facilities, and may have no formal systems and protocols for sample management and tracking. Microsampling methods, such as volumetric absorptive microsample (YAMS), dried blood spot (DBS), dried plasma spot (DPS), and capillary microsampling, may reduce the need for or eliminate the use of venipuncture for many assays. However, the difference in venous and capillary blood biomarkers and low sample volume may make these techniques difficult to adapt to some assays, such as complete blood count (CBC), cell-free circulating tumor DNA (ctDNA) liquid biopsies, and circulating free DNA (cfDNA) pathogen metagenomics. Thus, devices and method for simplifying phlebotomy, reduce variability, and improve process safety may increase harmonization, compliance, and reduce testing costs.

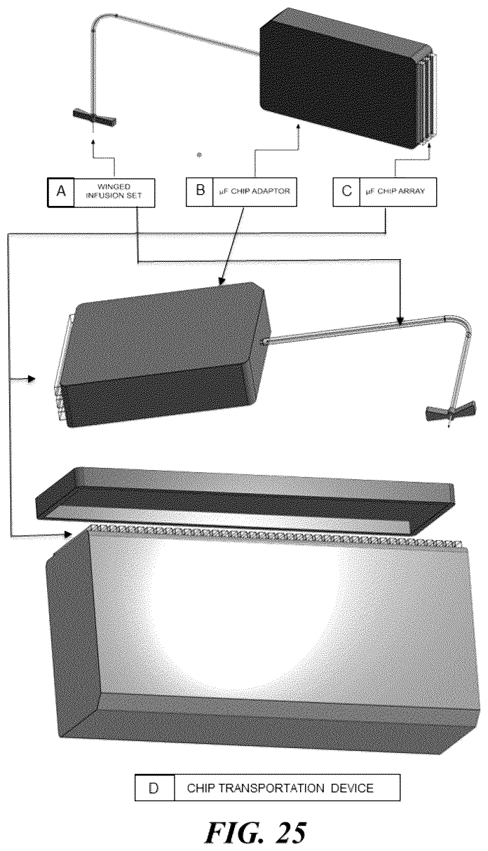

The sample collection device may comprise a microfluidic device for collection of bodily fluids (e.g., blood, urine, feces, etc.). The sample collection device may be used in tandem with an advance laboratory information management system (LIMS) for providing workflow in real-time. The LIMS may provide voice or gesture enabled protocol walkthrough and quality tracking. FIG. 25 shows an illustration of an example sample collection device. The sample collection device (e.g., blood collection device) may include a winged infusion set A, microfluidic chip adaptor and holder B, microfluidic collection chip C, chip transportation device D, or any combination thereof.

The device may include one inlet port or multiple inlet ports. In an example, the device includes a single inlet port. The inlet port may be fluidically connected to a needle configured to draw blood from a subject. Alternatively, or in addition to, the inlet port may be configured to collect whole blood samples, whole blood samples with plasma separation, plasma samples, bronchoalveolar lavage samples, urine sample, sputum samples, synovial fluid samples, liquified stool samples, tissue fluid samples, any other biological fluid sample, or any combination thereof. In an example, the inlet port is configured to collect blood. In an example, the inlet port is a winged infusion set configured for venipuncture. The winged infusion set may be a commercially available winged venal puncturing drawing syringe kit.

The inlet port may be configured to seal or may seal. The inlet port may be configured with a self-sealing plug. The inlet port may include a probe, needle, tube, or other device for collecting a bodily fluid. The device for collecting a bodily fluid may or may not be removable. In an example, the sample collection device comprises a needle and the needle may be removable. Upon removal of the needle, the inlet port may be sealed to prevent or reduce contamination.

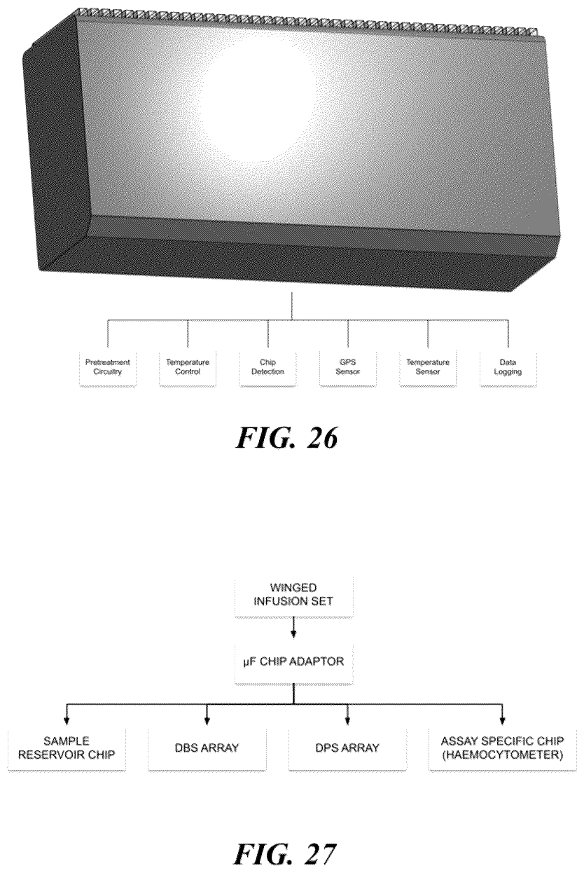

The sample collection device may include an adapter configured to hold or otherwise hold and transport the sample. The adapter may interface with one or more chips. The adapter may be configured to multiplex the sample into multiple chips (e.g., a chip array) held within the adapter. The adapter may permit the chips to be securely interfaced with the inlet port. The adapter may include pretreatment circuitry for pretreatment of the sample, temperature control, chip detection, a global position satellite (GPS) sensor, temperature sensor, data logger, or any combination thereof, as show in FIG. 26. The adapter may be configured to or may otherwise detect a chip, identify a chip, control a temperature of the chip, detect a location of the chip (e.g., via a global positioning satellite), log data, detect tampering, or any combination thereof. The device may be configured to or may otherwise track and monitor a sample once the sample is input into the device. A chip's function may range from sample reservoir with anticoagulant mixture to plasma separation to assay specific circuitry.

The adapter may provide an interface for straight needle draws or capillary collection. The adapter may hold and interface with a single type of chip or multiple types of chips. The chips may be different for different assays and collection types. The chips may be configured to or may otherwise hold the sample, provide buffers, reagents, or other additives to the sample, or any combination thereof. For example, and as shown in FIG. 27, the adapter may interface with chips comprising a sample reservoir, DBS array, DPS array, or another assay specific chip (e.g., hemocytometer). In an example, the chip comprises a sample reservoir and is configured for immediate plasma separation. Immediate plasma separation may reduce the risk of in vitro processes impacting biomarkers, such as cell lysis (e.g., gDNA contamination of cfDNA, intracellular potassium contamination, etc.) or in vitro metabolism (e.g., red blood cell glycolysis). In another example, the chip may comprise a DBS or DPS array. Incorporation of a DBS or DPS array may provide efficient sample storage and retrospective testing (e.g., for clinical research applications). In another example, the chip may comprise components for hemocytometry. A hemocytometry chip may include dilution, lysing, staining, counting chambers, or any combination thereof. The hemocytometry chip may be hydrostatically driven. The sample may be drawn into the chip and the assay prepared at the point of collection. The assay may then be analyzed by optical cytometry in the laboratory without additional sample preparation.

The adapter may include a processing drive. The processing drive may be configured for pneumatic control, temperature control, transportation, or any combination there. For example, for processes that may not be driven by hydrostatic pressure, a portable chip drive that may double as a transportation cold box may provide pneumatic and temperature control for sample collection and transport.

The device may include a single chip or multiple chips. In an example, the device includes at least two chips. The device may include 1, 2, 4, 6, 8, 10, 12, 15, 20, or more chips. The number of chips in the sample collection device may be altered or modified based on the application. In an example, the sample collection device has one or more chips. The device may divide the sample between the chips. The device may multiplex the sample into each chip in the device.

The sample collection device, as described herein, may simplify workflows for sample collection, transportation, storage, or any combination thereof for improved patient safety and comfort. Furthermore, the sample collection device may reduce the risk of operator injury, error, and cross contamination. The sample collection device may permit a reduction in sample volumes for various assays and simultaneous sample draws. This may reduce time of the draw, risk of nosocomial anemia, risk of contamination by eliminating order of draw, cognitive load on the phlebotomist to improve focus on safety and comfort, reagent consumption, resource use for transportation, and biohazardous waste. Additionally, immediate sample processing may reduce the risk of in vitro metabolic impact on biomarkers (e.g., red blood cell glycolysis), risk of cell lysis on biomarkers, turnaround time for testing, shipping and handling requirements, expensive equipment and personnel training for sample processing, operator variability and human error, or any combination thereof.

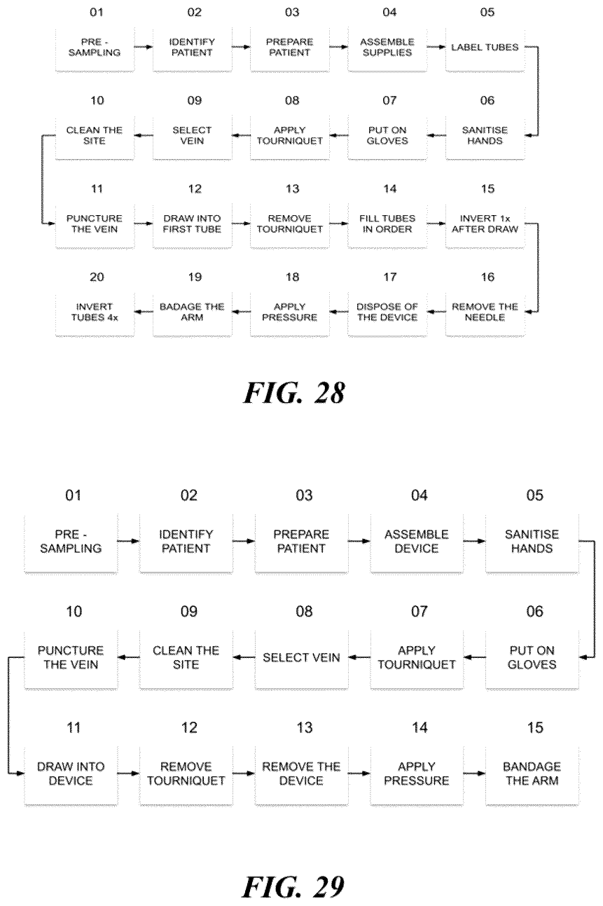

FIG. 28 shows example process flow guidelines from European Federation of Clinical Chemistry and Laboratory Medicine (EFLM) for venipuncture. The process flow may include pre-sampling, patient identification, patient reparation, assembly of supplies, labeling tubes, sanitizing hands, putting on gloves, applying the tourniquet, selecting the vein, cleaning the site, puncturing the vein, drain in the first tube, remove the tourniquet, fill tubes in order, invert one time after draw, remove the needle, dispose of device, apply pressure, bandage the draw site, and invert the tubes four more times. Workspace preparation for venipuncture may use a written protocol and planning. Materials to be used may be easily accessible and supplies may be used within the expiration date. Workspaces may be arranged such that the phlebotomist can reach the supplies without leaving the place of blood draw. Materials used for venipuncture may include a utility cart, blood collection trays, gloves, blood collection system with safety features, blood collection tubes, tourniquet, antiseptics, bandages, sharps bin, sample mixer, leak proof transportation bags, or any combination thereof.

Using the sample collection device described herein may simplify the process flow for sample collection (e.g., blood draw). For example, as shown in FIG. 29, sample collection via venipuncture may include pre-sampling, patient identification, patient preparation, assemble device, sanitize hands, put on gloves, apply tourniquet, select vein, clean the site, puncture the vein, draw into device, remove tourniquet, remove the device, apply pressure, and bandage the arm. Materials used for venipuncture may include the sample collection device, gloves, tourniquet, antiseptics, bandages, sharps bin, chip holder, or any combination thereof. The sample collection protocol and collected sample may be monitored and the monitored information may be provided to the LIMS.

In an example, the sample collector (e.g., phlebotomist) may prepare the patient for sample collection. The LIMS may prompt the sample collector through the sample collection interface to seat the patient comfortably for 15 minutes. During this time, the phlebotomist may be prompted to inform the patient of the procedure, address any questions, confirm that they understand and are comfortable, or any combination thereof. The sample collector may be prompted by sample collection interface to capture dietary and fasting compliance data, supplement usage, review current medication details, or any combination thereof. Medicine and supplement data may be collected by scanning barcodes from the medicine or supplement bottles and confirmed by the sample collector. If the bottles are not available, the sample collector interface may prompt the sample collector to confirm if certain medicines or supplements are being consumed based on the given test ordered. The LIMS may query the knowledge base and provide information if interaction that may lead to spurious results are detected. Based on the organizational guidelines either the sample may not be collected or the impacted test may be flagged and not preformed. Additional prompts may capture data, including, but no limited to, latex allergy, risk of vasovagal reaction, test specific factors such as female hormones on luteinizing hormone (LH), follicle-stimulating hormone (FSH), estradiol (E2), progesterone, etc., or any combination thereof. Select data may not be available or definitive at the time of sample collection. For example, a patient may not know if they have a latex allergy and, as the symptoms may not manifest immediately and reactions may not occur until up to eight hours after exposure, the LIMS may prompt a review from the sample collector at the end of the procedure to confirm symptoms and follow up with the patient if any and user feedback after the procedure.

The sample collector may assemble the sample collection device. Assembly may include connecting the chip holder to the infusion set. The chip's RFID tag may be scanned by the sample collector to map the chip and verify within the expiration date. A human readable label may then be printed using a portable label printer or manually written and attached. The sample collector may be prompted to sanitize their hands and confirm sanitization to continue. Voice interfacing may reduce the risk of contamination throughout the remainder of sample collection. The sample collector may be prompted to put on the appropriate personal protective equipment (e.g., gloves, mask, gown, etc.), confirm, and continue. In the case of a blood draw, the sample collector may be prompted to apply the tourniquet. As tourniquets may be a source of antibiotic resistant infections (e.g., MRSA), the sample collection interface may request confirmation that a fresh tourniquet is used to permit continuation of sampling. The sample collector (e.g., phlebotomist) may be prompted to select a vein and confirm the correct needle gauge is being used. The sample collecting interface may prompt the sample collector to sanitize the site with appropriate sanitizer for the test ordered and request confirmation of sanitization and that the site has dried before permitting continuation. Based on organizational policies, individual pre-soaked swabs may be used and may be scanned into the system as well. The sample collector may puncture the vein, draw into the device, remove the tourniquet, remove the device, apply pressure, and bandage the arm. These actions may be prompted by the sample interface with minimal data capture during the processes to ensure the sample collector's focus is on patient safety and comfort along with sample collector safety. Alternatively, or in addition to, a simple voice command may capture compliance and move to the next action. The sample collector may then be prompted to review the actions along with annotations if any to complete the workflow. Quality indicators may be captured through data input as well as timing information (e.g., if the time between clean site prompt and confirmation indicates improper site drying).

### Laboratory Information Management Systems and Related Methods

In an aspect, the present disclosure provides systems for processing a sample of a subject. The systems may include a computer server in communication with a plurality of user devices (e.g., 1, 2, 3, 4, 5, 6, 8, 10, 15, 20, or more user devices). The computer server may include (i) a database (e.g., knowledge base) for storing test information and clinical information (ii) a memory for storing a set of software instructions, and (iii) one or more computer processors configured to execute a set of software instructions to (a) receive, from a first (e.g., doctor, clinician, etc.) user a request for analysis of a sample, (b) request, from a second user (e.g., a patient or subject) device health of physiological information of the subject, (c) query the database (e.g., knowledge base) to (i) retrieve the test information and clinical information and (ii) use the test information and clinical information to generate pre-collection constraints and a sample collection protocol, (d) provide the pre-collection constraints to a user of the second user device, and (e) provide the sample collection protocol to a third user (e.g., technician, phlebotomist, etc.). The sample collection protocol may permit a sample collector to collect the sample for the test.

In another aspect, the present disclosure provides methods for processing a sample of a subject. The method may include providing a computer server in communication with a plurality of user devices (e.g., 1, 2, 3, 4, 5, 6, 8, 10, 15, 20, or more user devices). The computer server may include (i) a database (e.g., knowledge base) for storing test information and clinical information, (ii) a memory for storing a set of software instructions, and (iii) one or more computer processors to execute the set of software instructions. The computer server may receive, from a first user (e.g., doctor, clinician, etc.) device a request for analysis of a sample. The computer server may request health of physiological information from a second user (e.g., patient, subject, etc.) device. The computer server may query the database (e.g., knowledge base) to retrieve the test information and clinical information. The computer server may use the test information, the clinical information, or both to generate pre-collection constraints and a sample collection protocol. The computer server may provide the pre-collection constraints to the second user device and the sample collection protocol to a third user (e.g., technician, phlebotomist, etc.) device. The sample collection protocol may permit a sample collector to collect the sample from the subject for analysis of the sample.

In another aspect, the present disclosure provides systems for analyzing a sample of a subject. The system may include a computer server in communication with a user device and an analysis module configured to analyze a sample. The computer server may include a database (e.g., knowledge base) for storing test information, a memory for storing a set of software instructions, and one or more computer processors configured to execute a set of software instructions. The set of software instructions may permit the computer server to (a) receive, from the user (e.g., doctor, clinician, etc.) device, one or more input parameters, (b) query the database (e.g., knowledge base) to determine one or more testing conditions for analyzing the sample, (c) receive, from the analysis module, a status of the analysis module, (d) use the input parameters, testing conditions, and status of the analysis module to generate a testing schedule with a minimum testing turnaround time, and (d) provide the testing schedule to the analysis module to perform the analysis of the sample. The input parameters may include the type of analysis, number of tests to be performed, urgency of the analysis, or any combination thereof.

In another aspect, the present disclosure provides methods for analyzing a sample of a subject. The method may include providing a computer server in communication with a user (e.g., doctor, clinician, etc.) device and an analysis module. The computer server may include a database (e.g., knowledge base) for storing test information, a memory for storing a set of software instructions, and one or more computer processors configured to execute a set of software instructions. The software instructions may permit the computer server to receive one or more input parameters from the user device, query the database to determine one or more testing conditions for analyzing the sample, receive a status of the analysis module from the analysis module, use the one or more input parameters, one or more testing conditions, and the status of the analysis module to generate a testing schedule with a minimum testing turnaround time, and provide the testing schedule to the analysis module to analyze the sample. The input parameters may include type of analysis, number of tests to be performed, urgency of the test, or any combination thereof.

The system may include a hybrid cloud microservices or a plurality of hybrid cloud microservices. The system may run the set of microservices through the hybrid cloud and on-premise infrastructure. The hybrid cloud microservices may provide enterprise resource planning (ERP), customer relationship management (CRM), laboratory information management system (LIMS), laboratory information system (LIS), scientific data management system (SDMS), laboratory automation system (LAS), or any combination thereof. The laboratory information management system (LIMS) may include the laboratory information system (LIS). Alternatively, the laboratory information system may be separate from the laboratory information management system. Organizations may use any subset of the microservices to complement legacy systems or customize solutions to unique to the organization. For example, the hybrid cloud microservices may be used to reduce patient identification errors, increase the efficiency of data collection of clinically relevant information that may impact testing, reduce operator variability and cognitive load with real-time workflow walkthroughs, real-time collection and analysis of quality indicators for continuous improvement, or any combination thereof.

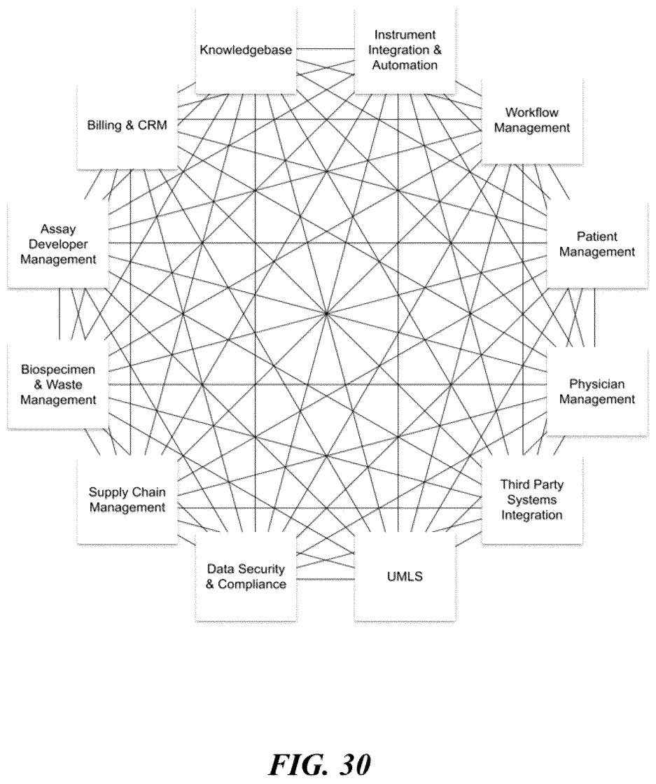

The microservices may be distributed over public and private clouds hosted globally, regionally, or on premise as per regulatory compliance and organizational guidelines. FIG. 30 shows an example of the LIMS, which may be provided by hybrid cloud-based and on-premise infrastructure. The microservices may be complementary to legacy systems or may be a customizable solution for a select application or use. The microservices may be used through even driven architecture. Application program interface (API), remote procedure call (RPC), and messaging interfaces may be integrated with legacy systems. The LIMS may include a knowledge base, instrument integration and automation, workflow management, patient management, physician management, third party systems integration, unified medical language systems (UMLS), data security and compliance, supply chain management, biospecimen and waste management, assay developer management, billing and customer relationship management, or any combination thereof.

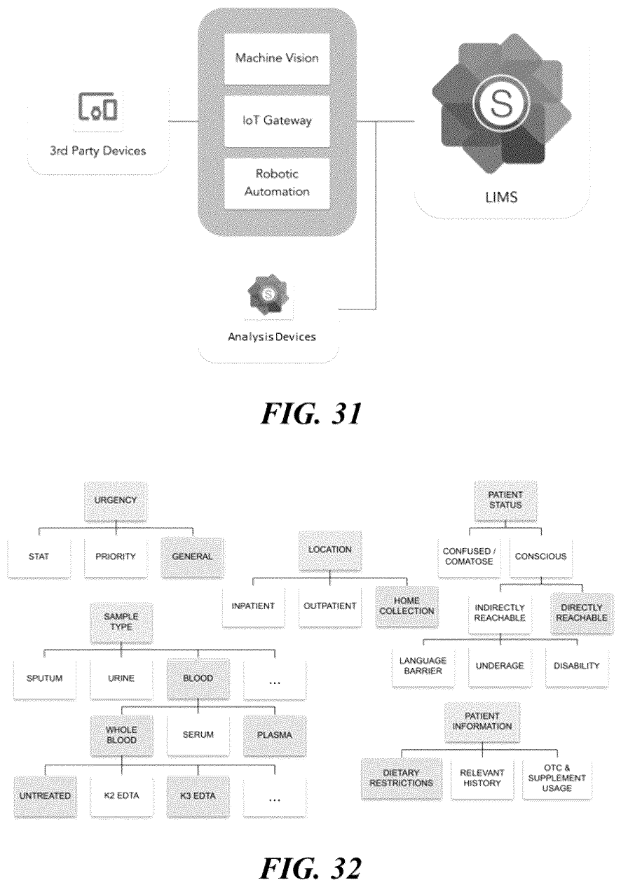

Instrumentation integration and automation may permit the integration of third party devices with the laboratory automation system by coupling to an internet of things (IoT) middleware gateway, machine vision, and robotic handling. FIG. 31 shows an example schematic of instrument integration and automation. Third party devices may be integrated with the LIMS and system devices (e.g., analysis system and other infrastructure) through an IoT middleware gateway, machine vision, and robotic handling. The IoT middleware gateway may interface with the devices (e.g., third party and system devices) and the communication protocols to permit the LAS to communicate with and control the system devices. For example, in the absence of machine to machine interfaces with the devices, machine vision and robotic control may be used to actuate and read results from the analysis system. In an example, the system is integrated with a top loading desktop thermocycler. The top loading desktop thermocycler may not include automation (e.g., an operator may interface to load, operate, and set cycle parameters), but may be integrated into the system through a chassis with a robotic arm or cartesian robot to open, load, and set thermocycle parameters. Machine vision may be used to confirm proper loading and parameter set up. The IoT middleware gateway may permit communication between the chassis and the LAS. In another example, the analysis system may include a thermal module designed for robotic integration. The thermal module may include a machine interface and, as such, the parameters may be set up through the thermal module's machine interface. The IoT middleware gateway may interface the thermal module to the LAS.

Workflow management may include test ordering and scheduling, sample management, run optimization, analysis, quality control, reporting, or any combination thereof. Tests may be ordered through a physician interface, (e.g., application or web interface) or through an integrated third party system. The physician may provide one or more input parameters. The input parameters may include type of analysis, number of tests, urgency of the testing, or any combination thereof. The testing conditions may include the number of operations required per assay, common sub-processes shared between assays, materials used for each assay, or any combination thereof.

The interface may include patient authentication details, such as patient identification for existing patients in the database or full name, address and contact details, identification for photo identification, additional details used for registering a patient as per regulatory or organizational guidelines, or any combination thereof. The request data may be validated against regulatory or organizational rules or both for compliance. The system may provide pre-collection constraints to a device of a doctor, clinician, technician, or patient. The pre-collection constraints may include dietary requirements, fasting requirements, or both dietary and fasting requirements. The test may be scheduled based on a matrix of parameters and weights as defined in a rules engine by the organization, patient availability, sample collector availability, or any combination thereof. Parameters may include urgency, sampling location, patient status, sample type, patient based constraints (e.g., dietary restrictions, relevant medical history, etc.), or any combination thereof. FIG. 32 schematically illustrates an example matrix of parameters for sample ordering and scheduling. Details of the clinician ordering the test may be provided to the system, such as full name and contact details. Patients may be informed of any pre-sampling constraints and sample collection may be scheduled using a patient interface (e.g., application, web interface, etc.), which may be natural language based and interfaced by voice, chat, or text messaging. Based on the rules defined, additional patient information such as over the counter (OTC) and supplement usage may be requested from the patient through the interface, confirmed by the sample collector at the time of collection, or both. The system may select and procure the relevant sample chips, generate a sampling protocol based on the tests requested and assign a sample collector.

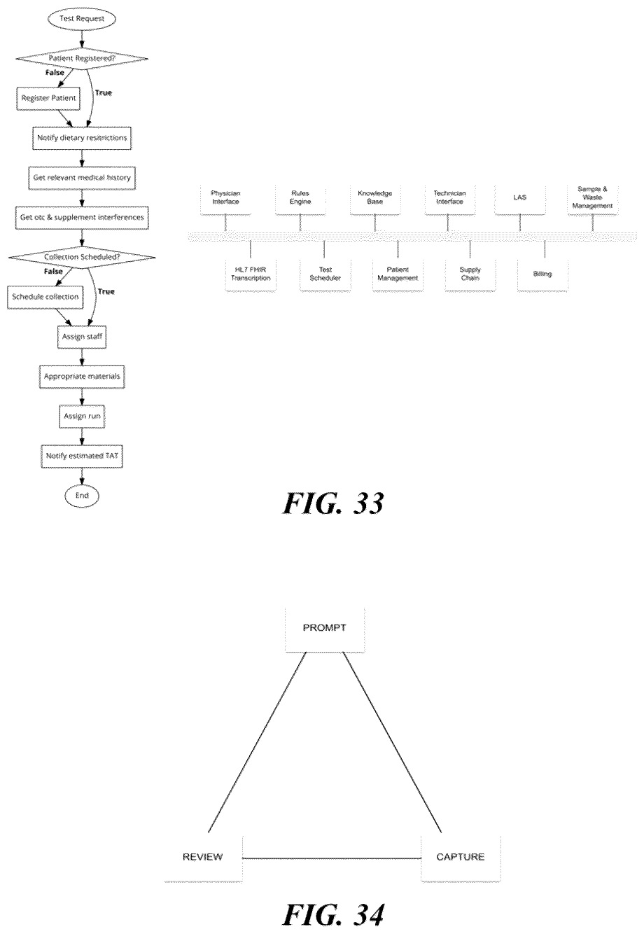

Sample management may include sampling, handling, transportation, or any combination thereof. Sampling, handling, and/or transportation may be tracked through user input, collection, storage, device transportation sensors, or any combination thereof to monitor sample viability and quality assurance tracking. FIG. 33 schematically illustrates an example process flow for analyzing a sample and LIMS integration. The clinician may request a test. If the patent is not registered, the system may prompt the clinician or patient to register. If the patient is registered or once the patient is registered, the system may notify the patient of dietary restriction, request relevant medical history, request OTC and supplement interferences. The dietary restriction may include dietary guidelines (e.g., no alcohol intake, no smoking, etc.) and fasting guidelines (e.g., how many hours before the test to fast). The patient may be notified of the dietary and fasting guidelines and reminded of the collection time and location by chatbots, push notifications, email, or text message. Additional reminders may be provided to the user (e.g., patient) to improve compliance. For example, the system may provide the patient with a 24 hour no alcohol reminder at a preset time before collection, reminder to not eat after a given time, reminder not to smoke the morning of a blood test, or any combination thereof. Relevant health history (e.g., health or physiological information) may include diagnosis, pre-diagnosis, current medication, other information relevant to testing, or any combination thereof. The health or physiological information of the subject may include medical history, over the counter medication usage, supplement usage, or any combination thereof. The system may be configured to alert a user (e.g., doctor, clinician, technician, patient, etc.) if a medication, over the counter medication, or supplement interferes with analysis of the sample.

The system may provide the impact of current medication on the requested test. If a patient's current medication is unavailable to the system, this information may be collected further downstream in the sample collection and processing workflow. If the collection is not scheduled, the system may prompt the patient or clinician to schedule the collection. The system may be configured to provide or may provide scheduling information to the doctor, clinician, patient, etc. device. The scheduling information may include sample collection date, sample collection time, location of the sample collection, personnel assigned to collect the sample, or any combination thereof. Once the collection is scheduled, the system may assign a sample collector, provide the materials to be used, provide sample collection workflow, assign the run, and notify the clinician, patient, or other party of the estimated turnaround time (TAT). The system may be configured to provide or may provide an estimated turnaround time from sample collection to receiving results of the analysis of the sample. The results may be provided to a device of the doctor, clinician, patient, or other involved party. Sample management may include the use of a physician interface, rules engine, knowledge base (e.g., database), technician (e.g., sample collector) interface, LAS, sample and waste management, health level 7 fast healthcare interoperability resources (HL7 FHIR) transcription, test scheduler, patient management, supply chain, billing, or any combination thereof.

At the time of sampling, the sample collector (e.g., technician) may use a sample collection interface. The sample collection interface may provide prompt, capture, and review cycles, as shown in FIG. 34, to increase workflow compliance, information capture, validation, or any combination thereof. The sample collection interface may be an application or web interface. The application may be interfaced through voice, gesture, touch, or a combination thereof and include visual and auditory feedback. For example, the application may prompt a phlebotomist (e.g., sample collector) to positively identify the patient. Based on regulatory and organizational guidelines, the phlebotomist may scan an approved photo identification card to capture the full name and other available patient details (e.g., date of birth, physical attributes, etc.). The application may request the phlebotomist review and confirm the accuracy of the information captured. Additional information, such as health insurance, may be similarly captured.

The sample collector (e.g., phlebotomist) may prepare the patient for sample collection. The LIMS may prompt the sample collector through the sample collection interface to seat the patient comfortably for fifteen minutes. During this time, the phlebotomist may be prompted to inform the patient of the procedure, address any questions, confirm that they understand and are comfortable, or any combination thereof. The sample collector may be prompted by sample collection interface to capture dietary and fasting compliance data, supplement usage, review current medication details, or any combination thereof. Medicine and supplement data may be collected by scanning barcodes from the medicine or supplement bottles and confirmed by the sample collector. If the bottles are not available, the sample collector interface may prompt the sample collector to confirm if certain medicines or supplements are being consumed based on the given test ordered. The LIMS may query the knowledge base and provide information if interaction that may lead to spurious results are detected. Based on the organizational guidelines either the sample may not be collected or the impacted test may be flagged and not preformed. Additional prompts may capture data such as latex allergy, risk of vasovagal reaction, test specific factors such as female hormones on luteinizing hormone (LH), follicle-stimulating hormone (FSH), estradiol (E2), progesterone, etc., or any combination thereof. Select data may not be available or definitive at the time of sample collection. For example, a patient may not know if they have a latex allergy and, as the symptoms may not manifest immediately or after an hour, the LIMS may prompt a review from the sample collector at the end of the procedure to confirm symptoms if any and user feedback after the procedure.

The sample collector may assemble the sample collection device. Assembly may include connecting the chip holder to the infusion set. The chip's ultrahigh frequency (UHF) tag may be scanned by the sample collector to map the chip and verify within the expiration date. A human readable label may then be printed using a portable label printer or manually written and attached. The sample collector may be prompted to sanitize their hands and confirm sanitization to continue. Voice interfacing may reduce the risk of contamination throughout the remainder of sample collection. The sample collector may be prompted to put on the appropriate person protective equipment (e.g., gloves, mask, gown, etc.), confirm, and continue. In the case of a blood draw, the sample collector may be prompted to apply the tourniquet. As tourniquets may be source of antibiotic resistant infections (e.g., MRSA), the sample collection interface may request confirmation that a fresh tourniquet is used to permit continuation of sampling. The sample collector (e.g., phlebotomist) may be prompted to select a vein and confirm the correct needle gauge is being used. The sample collecting interface may prompt the sample collector to sanitize the sit with appropriate sanitizer for the test ordered and request confirmation of sanitization and that the site has dried before permitting continuation. Based on organizational policies, individual pre-soaked swabs may be used and may be scanned into the system as well. The sample collector may puncture the vein, draw into the device, remove the tourniquet, remove the device, apply pressure, and bandage the arm. These actions may be prompted by the sample interface with minimal data capture during the processes to ensure the sample collector's focus is on patient safety and comfort along with sample collector safety. Alternatively, or in addition to, a simple voice command may capture compliance and move to the next action. The sample collector may then be prompted to review the actions along with annotations if any to complete the workflow. Quality indicators may be captured through data input as well as timing information (e.g., if the time between clean site prompt and confirmation indicates improper site drying).

The sample may be transported to the integrated system. The sample may be transported in the sample collection device (e.g., adapter and holder). The sample may be tracked by the LIMS during collection, transport, and loading into the analysis system. The system may optimize each run for the highest throughput and fastest turnaround time. The system may provide a status of the analysis module. The status of the analysis module may include the capacity of the analysis module, number of chip bays available, current operation status, estimated time for each test to be run, or any combination thereof. The system may be configured to or may accept mid-run emergency runs. The system may be configured such that analysis of additional samples are permitted to be added to an analysis queue during operation of the system. For example, the system may be running a method and an additional method may be added to the queue during system operation. The additional method may be added to the end of the queue (e.g., after all other tests have been completed) or at another place in the queue. For example, if the system is in the middle of a run and a clinician orders an urgent test, the system may recompute subsequent runs to compensate for the impact on the current run. Factors used to compute run optimization may include test urgency, assays to be run, submodule capacity, bays used per assay, common sub process (e.g., nucleic acid extraction, library preparation, etc.), time per sub process, and/or other processes defined by an organization's rules engine. For example, noninvasive prenatal testing (NIPT) assays and cctDNA assays using NGS may use a sample collection chip, cfDNA extraction chip, an assay specific library preparation chip, or any combination thereof. Therefore, the system may optimize the turn around time by running the similar processes in parallel for the NIPT and cctDNA assays.

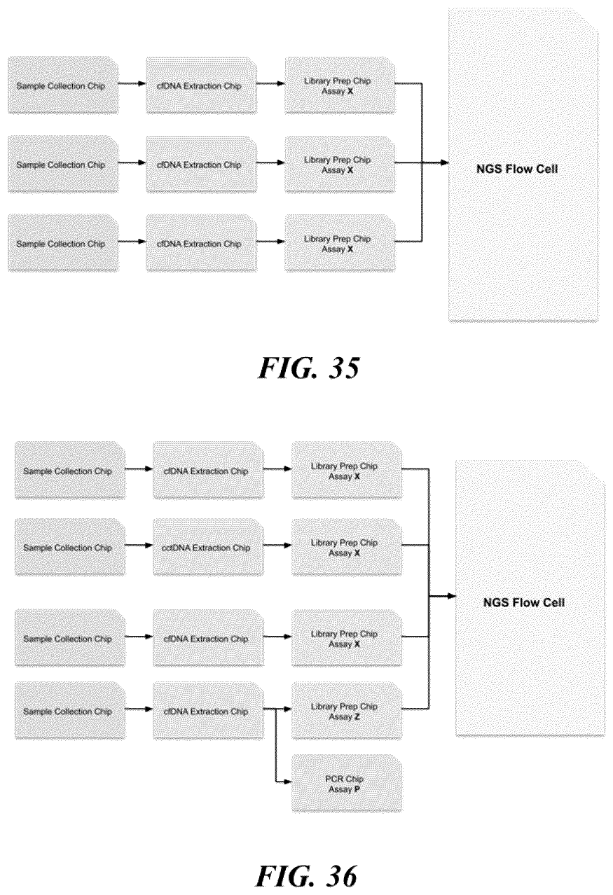

In an example, sample processing and analysis may include library preparation and NGS analysis. For example, FIG. 35 schematically illustrates example processing flow of an sample for NGS analysis. The library may be prepared, the samples chemically barcoded, and multiplexed into a single run on an NGS analyzer. Depending on the number of samples and organizational protocol, the samples may be sequences immediately or samples from multiple runs may be aggregated and run over an extended period of time on the NGS analyzer.

In another example, an emergency sample for suspected antimicrobial resistance (AMR) septicemia may be ordered. FIG. 36 schematically illustrates and example process flow for an example urgent test. If the sequencer is mid-run and does not have on demand capabilities, then the assay may not be performed until the run completes. If the sequencing is yet to be done, but the sample capacity of the sequencer is exhausted, some samples may be deferred for the emergency sample. Sample deferral may not be one-to-one as the high ratio of background DNA may swamp pathogenic cfDNA. If the sequencer is on demand, it may or may not be economically feasible to run a single sample per flow cell. In such cases, sequencing may be deferred until more sample are available and prepared, which may lead to delay. A rapid multiplexed nucleic acid amplification test (NAAT) (e.g., polymerase chain reaction, droplet digital polymerase chain reaction, loop mediated isothermal amplification, etc.) for detection of AMR genes may be simultaneously performed with library preparation. The NAAT may permit for timely treatment decisions while the NGS may provide a complete picture for diagnosis. The system may compute the optimum process based on the organizational policies defined in the rules engine. Manual override may provide for extenuating circumstances and be handled as per the protocol defined in the rules engine.

The system may be configured to alert a user (e.g., technician, doctor, clinician, etc.) when analysis of the sample has been completed. The method may include alerting a user when analysis of the sample has been completed.

The system may include a unified medical language system (UMLS). This service may transcribe information in and out of the system using unified codes, terms and vocabularies for interoperability with legacy systems, retrospective analysis, quality assurance, clinical studies across organization and regions, or any combination thereof. Standardized vocabularies may improve machine learning performance in generating and querying the knowledge base for timely and correct clinical outcomes.

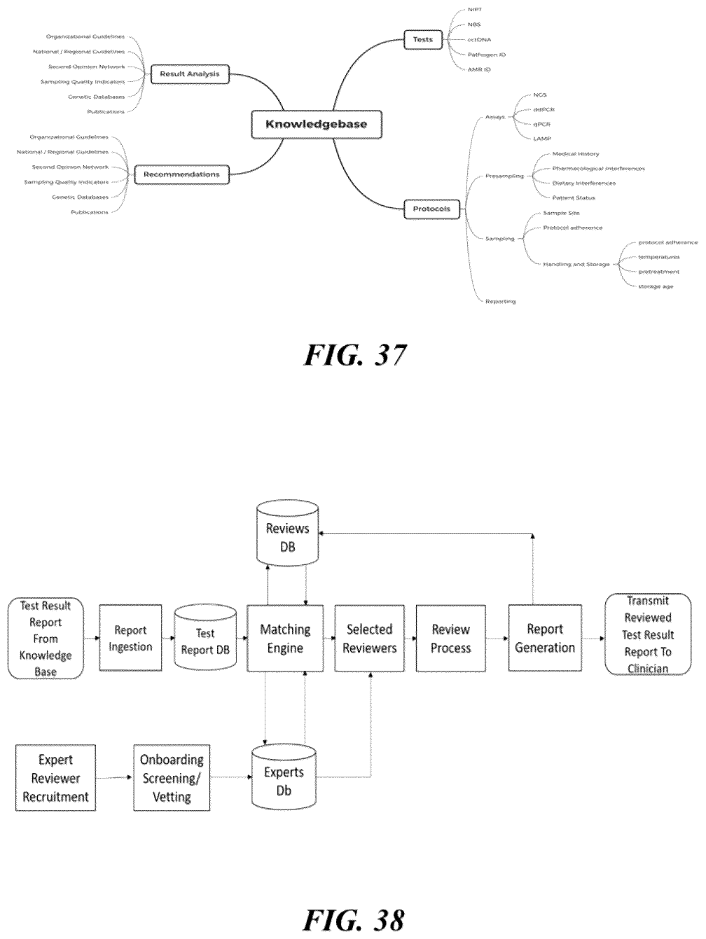

The results of the analysis may be provided to the user (e.g., technician, doctor, clinician, etc.). The system may include a database (e.g., knowledge base). The database (e.g., knowledge base) may comprise testing information, protocols for analysis, result analysis guidelines, recommendation guidelines, or any combination thereof. FIG. 37 schematically illustrates example architecture of a knowledge base. The database or knowledge base may be a knowledge graph comprising diagnostic tests, clinically relevant information, a second opinion network or authorized experts, or any combination thereof. The second opinion network may improve repeatability of testing, improve results analysis and interpretation, and provide recommendations for improved clinical outcomes. The results may further be provided to a network of authorized experts. A list of authorized experts may be stored in the database (e.g., knowledge base). The network of authorized experts may include an independent panel of verified experts. An expert from the network of authorized experts may provide recommendations based on the results of the analysis of the sample.

A bottleneck for clinical genetic testing and molecular diagnostics may be the review process for results by domain specific clinicians and experts. Many areas of the world may lack such health professionals who are able to interpret results and provide guidance. This issue may be solved by the second opinion review network, which may include a network of verified independent experts able to give their opinion, recommendation, and guidance for the clinician or physician that has ordered the test. The second opinion review network may be a useful decision-support tool to achieve a re-evaluation of a patient's case with consequent optimization of treatment and prognosis, which may permit avoidance of unnecessary surgery and costs. The network may include a wide panel of specialists from a variety of fields, including trained biologists and bioinformaticians who can support clinicians that have requested patient tests. The second opinion reviewer may analyze the clinical profile of the patient, the generic or molecular test report, knowledge base matched case studies and clinical literature, or any combination thereof to give guidance to the clinician or the physician of the patient. The network service may service multiple emergent clinical problems and may recruit in real-time a wide heterogeneous diagnostic panel of specialists simultaneously. The domain specialists selection may be based on personal knowledge, screening of individual CVs, publications, and specific expertise on selected cohorts of medical and surgical patients, including outcomes and represented institutions. A process of continuous screening and scouting of public and private centers and specialists may be implemented to satisfy the patient clinicians' requirements, on the basis of quality, scientific level, and field of specialty. The second opinion review service may be an option available to the clinician or physician ordering the test. The review may also be ordered after the clinician or physician has received an unreviewed test result report. FIG. 38 schematically illustrates an example process flow for the second opinion review network. A test report may be exported from the knowledge base and may be ingested by the LIMS to the test report database which provides the report to the matching engine. The matching engine may select reviewers, who review the process, generate a report, which is then delivered to the clinician. This cycle may repeat with multiple reviewers.

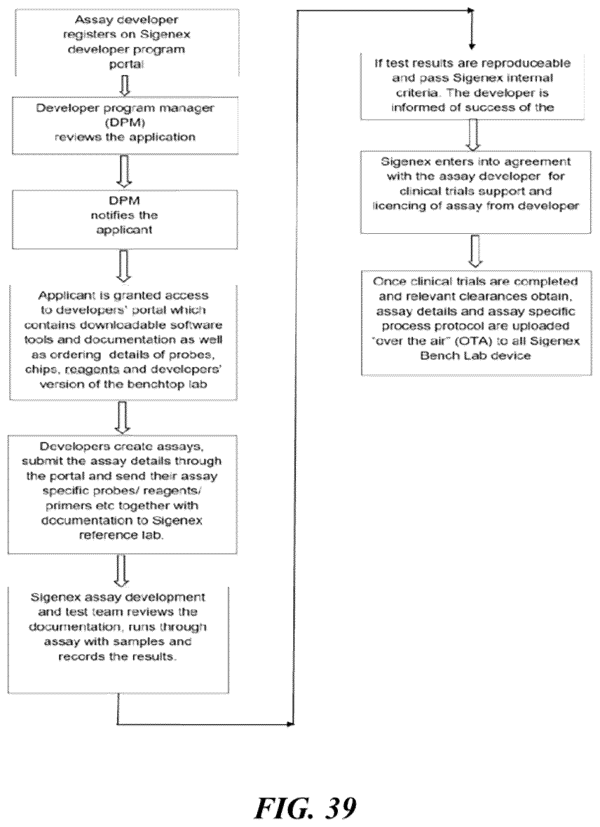

The system may be configured for assay development. For example, clinical trial protocols may include markers for determining eligibility, stratification, treatment assignment (e.g., integral markers), or any combination thereof. The assays used to determine these markers may not meet the standards used for clinical decision making. Therefore, predictive markers and robust methods to measure the markers may enable determination of integral markers. Towards this end, an assay developer program may be used to develop assays to measure and identify integral markers. The developer program may include a set of processes, software, and hardware tools to permit researchers to efficiently develop assays that may run on the analysis system described herein, thus permitting the co-development of targeted agents and predictive markers. FIG. 39 shows an example process flow for the development of assays on the analysis platform. An assay developer may register with the developer program portal. The developer program manager (DPM) may review the application and notify the applicant if access is granted. The applicant may be granted access to the developers portal, which may include downloadable software tools and documentation as well as ordering details of probes, chips, reagents, and developers version of the benchtop laboratory. The developers may create assays, submit the assay details through the portal and send the assay specific probes, reagents, primers, etc., together with documentation to a reference lab. The reference lab may have an assay development and test team that reviews the documentation, runs through the assay with samples and records the results. If the test results are reproducible and pass internal criteria, the developer is informed of the success. An agreement may be entered into with the assay developer for clinical trials support and licensing of the assay from the developer. Upon completion of clinical trials and relevant clearances have been obtained, the assay protocol may be uploaded to the knowledge base for use by others. The assay may then be made available on the platform post regulatory approvals.

### Computer Systems

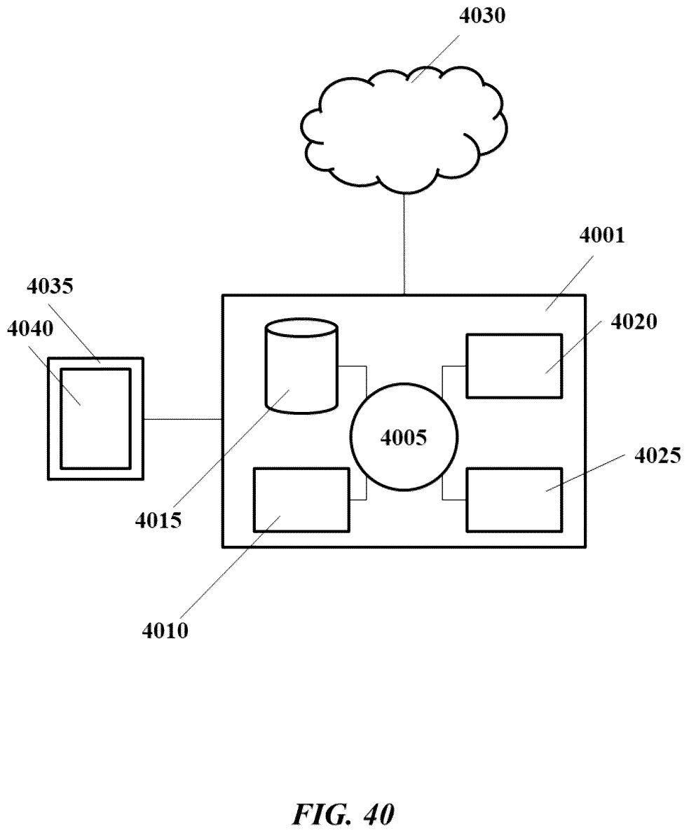

The present disclosure provides computer systems that are programmed to implement methods of the disclosure. FIG. 40 shows a computer system 4001 that is programmed or otherwise configured to control an analysis system or other sample processing device. The computer system 4001 can regulate various aspects of the analysis system of the present disclosure, such as, for example, regulating the processing module, thermal module, detection module, etc. The computer system 4001 can be an electronic device of a user or a computer system that is remotely located with respect to the electronic device. The electronic device can be a mobile electronic device.

The computer system 4001 includes a central processing unit (CPU, also “processor” and “computer processor” herein) 4005, which can be a single core or multi core processor, or a plurality of processors for parallel processing. The computer system 4001 also includes memory or memory location 4010 (e.g., random-access memory, read-only memory, flash memory), electronic storage unit 4015 (e.g., hard disk), communication interface 4020 (e.g., network adapter) for communicating with one or more other systems, and peripheral devices 4025, such as cache, other memory, data storage and/or electronic display adapters. The memory 4010, storage unit 4015, interface 4020 and peripheral devices 4025 are in communication with the CPU 4005 through a communication bus (solid lines), such as a motherboard. The storage unit 4015 can be a data storage unit (or data repository) for storing data. The computer system 4001 can be operatively coupled to a computer network (“network”) 4030 with the aid of the communication interface 4020. The network 4030 can be the Internet, an internet and/or extranet, or an intranet and/or extranet that is in communication with the Internet. The network 4030 in some cases is a telecommunication and/or data network. The network 4030 can include one or more computer servers, which can enable distributed computing, such as cloud computing. The network 4030, in some cases with the aid of the computer system 4001, can implement a peer-to-peer network, which may enable devices coupled to the computer system 4001 to behave as a client or a server.

The CPU 4005 can execute a sequence of machine-readable instructions, which can be embodied in a program or software. The instructions may be stored in a memory location, such as the memory 4010. The instructions can be directed to the CPU 4005, which can subsequently program or otherwise configure the CPU 4005 to implement methods of the present disclosure. Examples of operations performed by the CPU 4005 can include fetch, decode, execute, and writeback.

The CPU 4005 can be part of a circuit, such as an integrated circuit. One or more other components of the system 4001 can be included in the circuit. In some cases, the circuit is an application specific integrated circuit (ASIC).

The storage unit 4015 can store files, such as drivers, libraries and saved programs. The storage unit 4015 can store user data, e.g., user preferences and user programs. The computer system 4001 in some cases can include one or more additional data storage units that are external to the computer system 4001, such as located on a remote server that is in communication with the computer system 4001 through an intranet or the Internet.

The computer system 4001 can communicate with one or more remote computer systems through the network 4030. For instance, the computer system 4001 can communicate with a remote computer system of a user (e.g., physician, patent, sample collector, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 4001 via the network 4030.

Methods as described herein can be implemented by way of machine (e.g., computer processor) executable code stored on an electronic storage location of the computer system 4001, such as, for example, on the memory 4010 or electronic storage unit 4015. The machine executable or machine readable code can be provided in the form of software. During use, the code can be executed by the processor 4005. In some cases, the code can be retrieved from the storage unit 4015 and stored on the memory 4010 for ready access by the processor 4005. In some situations, the electronic storage unit 4015 can be precluded, and machine-executable instructions are stored on memory 4010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 4001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 4001 can include or be in communication with an electronic display 4035 that comprises a user interface (UI) 4040 for providing, for example, status of the analysis system to an operator. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 4005. The algorithm can, for example, direct the analysis system to analyze a sample according to a given protocol.

### List of Embodiments

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A system for processing a sample, comprising: an input module configured to accept one or more chips from a user, wherein at least one of said one or more chips comprises said sample; a processing module configured to transfer said one or more chips from said input module to one or more cartridges, wherein said one or more cartridges are configured to process said sample; and one or more computer processors operatively coupled to said input module and said processing module, wherein said one or more computer processors are individually or collectively programmed to (i) direct said processing module to transfer said one or more chips from said input module to said one or more cartridges and (ii) direct said one or more cartridges to process said sample.

Embodiment 2. The system of embodiment 1, wherein said system is configured as a closed system.

Embodiment 3. The system of embodiment 1 or 2, further comprising an alert module configured to monitor said system.

Embodiment 4. The system of any one of embodiments 1-3, wherein said alert module alerts a user if said processing module is operating outside of set operating parameters.

Embodiment 5. The system of any one of embodiments 1-3, wherein said alert module is configured to alert a user if said system has been opened or otherwise tampered with.

Embodiment 6. The system of any one of embodiments 1-5, wherein said system further comprises a tracking module configured to track said sample as it is processed by said system.

Embodiment 7. The system of any one of embodiments 1-6, wherein said one or more chips or said one or more cartridges comprise (i) at least one machine readable identifier configured to be tracked by said tracking module as said sample is processed by said system or (ii) at least one human readable identifier.

Embodiment 8. The system of any one of embodiments 1-7, further comprising a sequencing unit configured to sequence at least a portion of said sample.

Embodiment 9. The system of any one of embodiments 1-8, further comprising one or more detection modules configured to analyze said sample.

Embodiment 10. The system of any one of embodiments 1-9, wherein said system is coupled to a cloud server, and wherein said cloud server is configured to permit a user of said system to remotely monitor and control said system.

Embodiment 11. The system of any one of embodiments 1-10, wherein said processing module comprises one or more components selected from the group consisting of a liquid handling unit, a pneumatics unit, a temperature control unit, and a transfer unit.

Embodiment 12. The system of any one of embodiments 1-11, wherein said processing module is configured to transfer said sample from a first chip of said one or more chips to a second chip of said one or more chips.

Embodiment 13. The system of any one of embodiments 1-12, wherein said processing module is configured to (i) transfer a chip of said one or more chips from a first cartridge of said one or more cartridges to a second cartridge of said one or more cartridges or (ii) transfer said sample from said chip to one or more other chips.

Embodiment 14. The system of any one of embodiments 1-13, wherein said one or more chips comprise a plurality of ports sealed with a self-sealing material.

Embodiment 15. The system of embodiment 14, wherein said one or more chips are configured to be fluidically connected using disposable connectors configured to puncture said self-sealing material.

Embodiment 16. A method for processing a sample, comprising: providing a system comprising an input module that accepts one or more chips from a user and a processing module that transfers said one or more chips from said input module to one or more cartridges; providing one or more chips to said input module, wherein at least one of said one or more chips comprises said sample; using said processing module to transfer said one or more chips from said input module to one or more cartridges; and using said one or more cartridges to process said sample.

Embodiment 17. The method of embodiment 16, wherein said system is configured as a closed system.

Embodiment 18. The method of embodiment 16 or 17, further comprising using an alert module to monitor said system.

Embodiment 19. The method of any one of embodiments 16-18, wherein said alert module alerts a user if said processing module is operating outside of set operating parameters.

Embodiment 20. The method of any one of embodiments 16-18, wherein said alert module alerts a user if said system has been opened or otherwise tampered with.

Embodiment 21. The method of any one of embodiments 16-20, further comprising using a tracking module to track said sample as it is processed by said system.

Embodiment 22. The method of any one of embodiments 16-21, wherein said one or more chips or said one or more cartridges comprise (i) at least one machine readable identifier that is tracked by said tracking module as said sample is processed by said system or (ii) at least one human readable identifier.

Embodiment 23. The method of any one of embodiments 16-22, further comprising using a sequencing unit to amplify or sequence at least a portion of said sample.

Embodiment 24. The method of any one of embodiments 16-23, further comprising using one or more detection modules to analyze said sample.

Embodiment 25. The method of any one of embodiments 16-24, further comprising using a cloud server coupled to said system to remotely monitor and control said system.

Embodiment 26. The method of any one of embodiments 16-25, wherein said processing module comprises one or more components selected from the group consisting of a liquid handling unit, a pneumatics unit, a temperature control unit, and a transfer unit.

Embodiment 27. The method of any one of embodiments 16-26, wherein said processing module (i) transfers a chip of said one or more chips from a first cartridge of said one or more cartridges to a second cartridge of said one or more cartridges or (ii) transfers said sample from said chip to one or more other chips.

Embodiment 28. The method of any one of embodiments 16-27, wherein said one or more chips comprise a plurality of ports sealed with a self-sealing material.

Embodiment 29. The method of any one of embodiments 16-28, wherein said one or more chips are fluidically connected using disposable connectors that puncture said self-sealing material.

Embodiment 30. A system for analyzing a sample, comprising: a processing module configured to couple to one or more cartridges, wherein, during use, a cartridge of said one or more cartridges comprises at least a portion of said sample; a detection module comprising at least one sensor configured to analyze said at least said portion of said sample, wherein said at least one sensor comprises a variable pathlength spectrophotometer configured to be in optical communication with at least a portion of said cartridge comprising said at least said portion of said sample; and one or more computer processors operatively coupled to said processing module and said sensing module, wherein said one or more computer processors are individually or collectively programmed to (i) direct said processing module to couple to said one or more cartridges, (ii) direct said detection module to be in optical communication with said at least said portion of said cartridge comprising said at least said portion of said sample, and (iii) direct said detection module to analyze said sample.

Embodiment 31. The system of embodiment 30, wherein a pathlength of said variable pathlength spectrophotometer is configured to vary from about 0.01 millimeters (mm) to 0.2 millimeters.

Embodiment 32. The system of embodiment 30 or 31, wherein said system is configured as a closed system.

Embodiment 33. The system of any one of embodiments 30-32, further comprising an alert module configured to monitor said system.

Embodiment 34. The system of any one of embodiments 30-33, wherein said alert module alerts a user if said processing module is operating outside of set operating parameters.

Embodiment 35. The system of any one of embodiments 30-33, wherein said alert module is configured to alert a user if said system has been opened or otherwise tampered with.

Embodiment 36. The system of any one of embodiments 30-35, wherein said system further comprises a tracking module configured to track said sample as it is processed by said system.

Embodiment 37. The system of any one of embodiments 30-36, wherein said one or more chips or said one or more cartridges comprise (i) at least one machine readable identifier configured to be tracked by said tracking module as said sample is processed by said system or (ii) at least one human readable identifier.

Embodiment 38. The system of any one of embodiments 30-37, further comprising a sequencing unit configured to sequence at least a portion of said sample.

Embodiment 39. The system of any one of embodiments 30-38, further comprising one or more detection modules configured to analyze said sample.

Embodiment 40. The system of any one of embodiments 30-39, wherein said system is coupled to a cloud server, and wherein said cloud server is configured to permit a user of said system to remotely monitor and control said system.

Embodiment 41. The system of any one of embodiments 30-40, wherein said processing module comprises one or more components selected from the group consisting of a liquid handling unit, a pneumatics unit, a temperature control unit, and a transfer unit.

Embodiment 42. The system of any one of embodiments 30-41, wherein said one or more chips comprise a plurality of ports sealed with a self-sealing material.

Embodiment 43. The system of any one of embodiments 30-42, wherein said one or more chips are configured to be fluidically connected using disposable connectors configured to puncture said self-sealing material.

Embodiment 44. A method for analyzing a sample, comprising: providing a system comprising a processing module that couples to one or more cartridges and a detection module that analyzes said sample; coupling said processing module to said one or more cartridges, wherein a cartridge of said one or more cartridges comprises at least a portion of said sample; positioning said detection module comprising a variable pathlength spectrophotometer in optical communication with at least a portion of said cartridge comprising said at least said portion of said sample; and said detection module to analyze said at least said portion of said sample.

Embodiment 45. The method of embodiment 44, wherein a pathlength of said variable pathlength spectrophotometer varies from about 0.01 millimeters (mm) to 0.2 millimeters.

Embodiment 46. The method of embodiment 44 or 45, wherein said system is configured as a closed system.

Embodiment 47. The method of any one of embodiments 44-46, further comprising using an alert module to monitor said system.

Embodiment 48. The method of any one of embodiments 44-47, wherein said alert module alerts a user if said processing module is operating outside of set operating parameters.

Embodiment 49. The method of any one of embodiments 44-47, wherein said alert module alerts a user if said system has been opened or otherwise tampered with.

Embodiment 50. The method of any one of embodiments 44-49, further comprising using a tracking module to track said sample as it is processed by said system.

Embodiment 51. The method of any one of embodiments 44-50, wherein said one or more chips or said one or more cartridges comprise (i) at least one machine readable identifier that is tracked by said tracking module as said sample is processed by said system or (ii) at least one human readable identifier.

Embodiment 52. The method of any one of embodiments 44-51, further comprising using a sequencing unit to amplify or sequence at least a portion of said sample.

Embodiment 53. The method of any one of embodiments 44-52, further comprising using one or more detection modules configured to analyze said sample.

Embodiment 54. The method of any one of embodiments 44-53, further comprising using a cloud server coupled to said system to remotely monitor and control said system.

Embodiment 55. The method of any one of embodiments 44-54, wherein said processing module comprises one or more components selected from the group consisting of a liquid handling unit, a pneumatics unit, a temperature control unit, and a transfer unit.

Embodiment 56. A system for controlling a temperature of a sample, comprising: a processing module configured to couple to one or more cartridges, wherein, during use, a cartridge of said one or more cartridges comprises at least a portion of said sample; a thermal module configured to control a temperature of at least a portion of said cartridge of said one or more cartridges comprising said at least said portion of said sample, wherein said thermal module comprises a pneumatic temperature control unit configured to be in thermal communication with said at least said portion of said cartridge; and one or more computer processors operatively coupled to said processing module and said thermal module, wherein said one or more computer processors are individually or collectively programmed to (i) direct said processing module to couple to said one or more cartridges, (ii) direct said thermal module to be in thermal communication with said at least said portion of said cartridge, and (iii) direct said thermal module to control said temperature of said at least said portion of said cartridge, thereby controlling a temperature of said at least said portion of said sample.

Embodiment 57. The system of embodiment 56, wherein said thermal module is configured to provide contactless temperature control.

Embodiment 58. The system of embodiment 56 or 57, wherein said thermal module comprises a Rank-Hilsch vortex tube.

Embodiment 59. The system of any one of embodiments 56-58, wherein said system is configured as a closed system.

Embodiment 60. The system of any one of embodiments 56-59, further comprising an alert module configured to monitor said system.

Embodiment 61. The system of any one of embodiments 56-60, wherein said alert module alerts a user if said processing module is operating outside of set operating parameters.

Embodiment 62. The system of any one of embodiments 56-60, wherein said alert module is configured to alert a user if said system has been opened or otherwise tampered with.

Embodiment 63. The system of any one of embodiments 56-62, wherein said system further comprises a tracking module configured to track said sample as it is processed by said system.

Embodiment 64. The system of any one of embodiments 56-63, wherein said one or more chips or said one or more cartridges comprise (i) at least one machine readable identifier configured to be tracked by said tracking module as said sample is processed by said system or (ii) at least one human readable identifier.

Embodiment 65. The system of any one of embodiments 56-64, further comprising a sequencing unit configured to sequence at least a portion of said sample.

Embodiment 66. The system of any one of embodiments 56-65, further comprising one or more detection modules configured to analyze said sample.

Embodiment 67. The system of any one of embodiments 56-66, wherein said system is coupled to a cloud server, and wherein said cloud server is configured to permit a user of said system to remotely monitor and control said system.

Embodiment 68. The system of any one of embodiments 56-67, wherein said processing module comprises one or more components selected from the group consisting of a liquid handling unit, a pneumatics unit, a temperature control unit, and a transfer unit.

Embodiment 69. The system of any one of embodiments 56-68, wherein said one or more chips comprise a plurality of ports sealed with a self-sealing material.

Embodiment 70. The system of any one of embodiments 56-69, wherein said one or more chips are configured to be fluidically connected using disposable connectors configured to puncture said self-sealing material.

Embodiment 71. A method for controlling a temperature of a sample, comprising: providing a system comprising a processing module that couples to one or more cartridges and a thermal module that controls a temperature of said sample; coupling said processing module to said one or more cartridges, wherein a cartridge of said one or more cartridges comprises at least a portion of said sample; positioning said cartridge such that at least a portion of said cartridge is in thermal communication with said thermal module, wherein said thermal module comprises a pneumatic temperature control unit; and using said thermal module to control a temperature of at least said portion of said cartridge, thereby controlling said temperature of said at least said portion of said sample.

Embodiment 72. The method of embodiment 71, wherein said thermal module provides contactless temperature control.

Embodiment 73. The method of embodiment 71 or 72, wherein said thermal module comprises a Rank-Hilsch vortex tube.

Embodiment 74. The method of any one of embodiments 71-73, wherein said system is configured as a closed system.

Embodiment 75. The method of any one of embodiments 71-74, further comprising using an alert module to monitor said system.

Embodiment 76. The method of any one of embodiments 71-75, wherein said alert module alerts a user if said processing module is operating outside of set operating parameters.

Embodiment 77. The method of any one of embodiments 71-75, wherein said alert module alerts a user if said system has been opened or otherwise tampered with.

Embodiment 78. The method of any one of embodiments 71-77, further comprising using a tracking module to track said sample as it is processed by said system.

Embodiment 79. The method of any one of embodiments 71-78, wherein said one or more chips or said one or more cartridges comprise (i) at least one machine readable identifier that is tracked by said tracking module as said sample is processed by said system or (ii) at least one human readable identifier.

Embodiment 80. The method of any one of embodiments 71-79, further comprising using a sequencing unit to amplify or sequence at least a portion of said sample.

Embodiment 81. The method of any one of embodiments 71-80, further comprising using one or more detection modules configured to analyze said sample.

Embodiment 82. The method of any one of embodiments 71-81, further comprising using a cloud server coupled to said system to remotely monitor and control said system.

Embodiment 83. The method of any one of embodiments 71-82, wherein said processing module comprises one or more components selected from the group consisting of a liquid handling unit, a pneumatics unit, a temperature control unit, and a transfer unit.

Embodiment 84. A device for processing a sample, comprising: a cartridge comprising one or more bays, wherein a bay of said one or more bays is configured to removably hold a chip, and wherein said bay comprises a first pattern of contact points, wherein said chip comprises a second pattern of contact points that are complementary to said first pattern of contact points.

Embodiment 85. The device of embodiment 84, wherein said cartridge is configured such that said bay is configured to removably couple to more than one type of chip.

Embodiment 86. The device of embodiment 84 or 85, wherein said chip is configured to be transferable from said cartridge to another cartridge.

Embodiment 87. The device of any one of embodiments 84-86, wherein said chip is configured to process said sample.

Embodiment 88. The device of any one of embodiments 84-87, wherein said chip is configured to perform at least one function during processing of said sample.

Embodiment 89. The device of any one of embodiments 84-88, wherein said at least one function is selected from the group consisting of extracting nucleic acid from said sample, library preparation, sequencing said sample, separating components from said sample, and performing an assay on said sample.

Embodiment 90. The device of any one of embodiments 84-89, wherein said cartridge comprises mesofluidic circuitry and macro-sized contacts and said chip comprises microfluidic circuitry and micro-sized contacts, and wherein said first pattern of contact points and said second pattern of contact points are configured to provide an interface between said macro-sized contacts and said micro-sized contacts.

Embodiment 91. The device of any one of embodiments 84-90, wherein said cartridge comprises a standard interface such that said cartridge is capable of performing more than one type of sample processing.

Embodiment 92. The device of any one of embodiments 84-91, wherein said cartridge is configured to analyze a sample via exchanging said chip for another chip.

Embodiment 93. The device of any one of embodiments 84-92, wherein said cartridge or said chip comprises an authentication unit configured for validation and tracking.

Embodiment 94. The device of any one of embodiments 84-93, wherein said authentication unit provides cryptographic security.

Embodiment 95. The device of any one of embodiments 84-94, wherein said cartridge comprises one or more members selected from the group consisting of pneumatics, transducers, actuators, sensors, micropumps, pressure generators, regulators, solenoid valves, electromagnets, temperature sensors, energy storage units, and electronic circuitry.

Embodiment 96. The device of any one of embodiments 84-95, wherein a contact of said first pattern of contacts and said second pattern of contacts is configured to provide one or more of power, electronic communication, pneumatic communication, electromagnetic communication, or any combination thereof to said chip.

Embodiment 97. The device of any one of embodiments 84-96, wherein said cartridge comprises a memory and wherein said memory stores an interface protocol or driver for said chip.

Embodiment 98. The device of any one of embodiments 84-97, wherein said chip comprises a self-sealing material, and wherein said cartridge comprises one or more needles to penetrate said self-sealing material.

Embodiment 99. A device for collecting a sample, comprising: an inlet port configured to collect a sample from a subject; one or more chips in fluid communication with said inlet port; and an adapter in fluid communication with said inlet port and said one or more chips, wherein said adapter is configured to direct said sample from one or more mesofluidic channels of said inlet port to one or more microfluidic channels of said one or more chips.

Embodiment 100. The device of embodiment 99, wherein said inlet port is fluidically connected to a needle configured to draw blood from a subject.

Embodiment 101. The device of embodiment 99 or 100, wherein said inlet port is configured to seal.

Embodiment 102. The device of any one of embodiments 99-101, wherein said device comprises at least two chips, and wherein said adapter is configured to multiplex said sample into each chip of said at least two chips.

Embodiment 103. The device of any one of embodiments 99-102, wherein said adapter is configured to transport said sample.

Embodiment 104. The device of any one of embodiments 99-103, wherein said adapter is configured for one or more of chip detection, chip identification, temperature control, temperature detection, location detection, data logging, tamper detection, or any combination thereof.

Embodiment 105. The device of any one of embodiments 99-104, wherein said device is configured to track and monitor said sample once said sample is input into said device.

Embodiment 106. The device of any one of embodiments 99-105, wherein said device is self-sealing.

Embodiment 107. The device of any one of embodiments 99-106, wherein a chip of said one or more chips is configured to (i) hold said sample or (ii) provide buffers, reagents, or other additives to said sample.

Embodiment 108. A system for processing a sample of a subject, comprising: a computer server in communication with a plurality of user devices, wherein said computer server comprises a (i) a database for storing test information and clinical information, (ii) a memory for storing a set of software instructions, and (iii) one or more computer processors configured to execute said set of software instructions to: receive, from a first user device, a request for analysis of said sample; request, from a second user device, health or physiological information of said subject; query said database to (i) retrieve said test information and said clinical information and (ii) use said test information and said clinical information to generate pre-collection constraints and a sample collection protocol; provide said pre-collection constraints to a user of said second user device; and provide said sample collection protocol to a third user device, wherein said sample collection protocol permits a sample collector to collect said sample for said test.

Embodiment 109. The system of embodiment 108, wherein said health or physiological information of said subject is selected from the group consisting of medical history, over the counter medication usage, supplement usage, and combinations thereof.

Embodiment 110. The system of embodiment 108 or 109, wherein said system is configured to alert said first user device or said second user device if a medication, over the counter medication, or supplement interferes with said analysis of said sample.

Embodiment 111. The system of any one of embodiments 108-110, wherein said system is further configured to provide an estimated turnaround time from sample collection to receiving results of said analysis of said sample to said first user device or said second user device.

Embodiment 112. The system of any one of embodiments 108-111, wherein said system is further configured to provide scheduling information to said first user device, said second user device, or said third user device.

Embodiment 113. The system of any one of embodiments 108-112, wherein said scheduling information comprises sample collection date, sample collection time, location of sample collection, personnel assigned to collect said sample, or any combination thereof.

Embodiment 114. The system of any one of embodiments 108-113, wherein said pre-collection constraints comprise dietary requirements or fasting requirements.

Embodiment 115. The system of any one of embodiments 108-114, wherein said system is configured to provide reminders to said second user device regarding said pre-collection constraints.

Embodiment 116. The system of any one of embodiments 108-115, wherein said sample collection protocol comprises materials used for sample collection or sample collection workflow.

Embodiment 117. The system of any one of embodiments 108-116, wherein said system is further configured to prompt a sample collection personnel to positively identify a subject prior to sample collection.

Embodiment 118. A method for processing a sample of a subject, comprising: providing a computer server in communication with a plurality of user devices, wherein said computer server comprises (i) a database for storing a test information and clinical information (ii) a memory for storing a set of software instructions, and (iii) one or more computer processors to execute said set of software instructions; receiving from a first user device of said plurality of user devices a request for analysis of said sample; requesting health or physiological information of said subject from a second user device of said plurality of user devices; querying said database to retrieve said test information and said clinical information; using said test information and said clinical information to generate pre-collection constraints and a sample collection protocol; providing said pre-collection constraints to said second user device; and providing said sample collection protocol to a third user device of said plurality of user devices, wherein said sample collection protocol permits a sample collector to collect said sample of said subject for analysis of said sample.

Embodiment 119. The method of embodiment 118, wherein said health or physiological information is selected from the group consisting of patent medical history, over the counter medication usage, supplement usage, and combinations thereof.

Embodiment 120. The method of embodiment 118 or 119, further comprising alerting said first user device or said second user device if a medication, over the counter medication, or supplement interferes with said analysis of said sample.

Embodiment 121. The method of any one of embodiments 118-120, further comprising providing an estimated turnaround time from sample collection to providing results of said analysis of said sample to said first user device or said second user device.

Embodiment 122. The method of any one of embodiments 118-121, further comprising providing scheduling information to said first user device, said second user device, or said third user device.

Embodiment 123. The method of any one of embodiments 118-122, wherein said scheduling information comprises sample collection date, sample collection time, location of sample collection, personnel assigned to collect said sample, or any combination thereof.

Embodiment 124. The method of any one of embodiments 118-123, wherein said pre-collection constraints comprise dietary requirements or fasting requirements.

Embodiment 125. The method of any one of embodiments 118-124, further comprising providing reminders to said second user device regarding said pre-collection constraints.

Embodiment 126. The method of any one of embodiments 118-125, wherein said sample collection protocol comprises materials used for sample collection or sample collection workflow.

Embodiment 127. The method of any one of embodiments 118-126, further comprising prompting a sample collection personnel to positively identify a subject prior to sample collection.

Embodiment 128. A system for analyzing a sample of a subject, comprising: a computer server in communication with a user device and an analysis module configured to analyze said sample, wherein said computer server comprises a (i) a database for storing test information, (ii) a memory for storing a set of software instructions, and (iii) one or more computer processors configured to execute said set of software instructions to: receive, from said user device, one or more input parameters, wherein said one or more input parameters comprise type of analysis and number of tests to be performed; query said database to determine one or more testing conditions for performing said analysis of said sample; receive, from said analysis module, a status of said analysis module; use said one or more input parameters, said one or more testing conditions, and said status of said analysis module to generate a testing schedule with a minimum testing turnaround time; and provide said testing schedule to said analysis module to perform said analysis of said sample.

Embodiment 129. The system of embodiment 128, wherein said database further comprises protocols for said analysis, result analysis guidelines, recommendation guidelines, or any combination thereof.

Embodiment 130. The system of embodiment 128 or 129, wherein said one or more input parameters further comprises testing urgency.

Embodiment 131. The system of any one of embodiments 128-130, wherein said one or more testing conditions comprises a number of operations required per assay, common sub-processes shared between assays, or materials required for each assay.

Embodiment 132. The system of any one of embodiments 128-131, wherein said status of said analysis module comprises capacity of said analysis module, number of chip bays available, current operation status, or estimated time for each test to be run.

Embodiment 133. The system of any one of embodiments 128-132, wherein said system is configured such that analysis of additional samples is permitted to be added to an analysis queue during operation of said system.

Embodiment 134. The system of any one of embodiments 128-133, wherein said system is further configured to alert said user device when said analysis of said sample is complete.

Embodiment 135. The system of any one of embodiments 128-134, wherein said system is further configured to provide results of said analysis to said user device.

Embodiment 136. The system of any one of embodiments 128-135, wherein said system is further configured to provide results of said analysis to a network of authorized experts, wherein a list of authorized experts is stored in said database.

Embodiment 137. The system of any one of embodiments 128-136, wherein said network of authorized experts comprise an independent panel of verified experts.

Embodiment 138. The system of any one of embodiments 128-136, wherein said system is further configured to permit said network of authorized experts to provide recommendations based on said results of said analysis of said sample.

Embodiment 139. A method for analyzing a sample of a subject, comprising: providing a computer server in communication with a user device and an analysis module, wherein said computer server comprises (i) a database for storing test information (ii) a memory for storing a set of software instructions, and (iii) one or more computer processors configured to execute said set of software instructions; receiving one or more input parameters from said user device, wherein said one or more input parameters comprise type of analysis and number of tests to be performed; querying said database to determine one or more testing conditions for performing said analysis of said sample; receiving a status of said analysis module from said analysis module; using said one or more input parameters, said one or more testing conditions, and said status of said analysis module to generate a testing schedule with a minimum testing turnaround time; and providing said testing schedule to said analysis module to perform said analysis of said sample.

Embodiment 140. The method of embodiment 139, wherein said database further comprises protocols for said analysis, result analysis guidelines, recommendation guidelines, or any combination thereof.

Embodiment 141. The method of embodiment 139 or 140, wherein said one or more input parameters further comprises testing urgency.

Embodiment 142. The method of any one of embodiments 139-141, wherein said one or more testing conditions comprises a number of operations required per assay, common sub-processes shared between assays, or materials required for each assay.

Embodiment 143. The method of any one of embodiments 139-142, wherein said status of said analysis module comprises capacity of said analysis module, number of chip bays available, current operation status, or estimated time for each test to be run.

Embodiment 144. The method of any one of embodiments 139-143, further comprising permitting additional analytical tests to be added to an analysis testing queue during operation of said system.

Embodiment 145. The method of any one of embodiments 139-144, further comprising alerting said user device when said analysis of said sample is complete.

Embodiment 146. The method of any one of embodiments 139-145, further comprising providing results of said analysis of said sample to said user device.

Embodiment 147. The method of any one of embodiments 139-146, further comprising providing results of said analysis of said sample to a network of authorized experts, wherein a list of said authorized experts is stored in said database.

Embodiment 148. The method of any one of embodiments 139-147, wherein said network of authorized experts comprise an independent panel of verified experts.

Embodiment 149. The method of any one of embodiments 139-147, further comprising permitting said network of authorized experts to provide recommendations based on said results of said analysis of said sample.

### EXAMPLES

### Example 1: Outpatient Process Flow

A patient (e.g., subject) may visit a doctor or clinician, who may examine the patient. The doctor or clinician may decide to run a specific test (e.g., genetic test). The doctor may log into a test ordering interface connected to the Laboratory Information Management System (LIMS) housed inside the analysis system. The doctor may enter the patient identification which may be transferred to the LIMS. The LIMS may review the inventory database to determine if the test consumables are in stock in the lab room. If the consumables are not in stock, the doctor may place an order for the consumables. If the consumables are in stock, the doctor may confirm the order for the test. The LIMS may send the test order alert to a nurse, phlebotomist, or other sample collector via a sample collection, tracking, and meta data application. The sample collector may check the ordered test information, including the type of sample to be collected and device for collecting the sample. The sample collector may select a suitable time and location or provide time and location options for the patient to select. The LIMS may send a message to the patient regarding the time and location(s) for sample collection. The patient may select the time and location and the LIMS may provide the confirmation to the sample collector.

At the scheduled time, the patient may arrive at the sampling location and the sample collector may confirm the identity of the patient. The sample collector may select the designated sampling device, which may be a sealed package, and scan the radiofrequency identifier (RFID) tag attached to the package. The RFID information may be matched to records in the LIMS and records of the sample collection packs, including serial number of the sample collection tube or container and information regarding reagents, buffers, other chemicals within the tube or container, sample extraction tool, etc. may be generated. The sample collector may collect the sample and scan the identification number of the sample tube or container using an RFID reader. The identification number may be provided to the LIMS and added to the unique test order entry, which may include the patient identification, test ordered, sample collection package identification, sample container unique identification number, or any combination thereof. The sample collector may collect additional meta data from the patient, including alcohol use prior to sampling, smoking, medication, diet, location from which the sample is taken, or any combination thereof and the meta data may be sent to the LIMS and added to the test record.

### Example 2: Inpatient Process Flow

A doctor or specialist may decide to run a specific test (e.g., genetic test). The doctor may log into a test ordering interface connected to the Laboratory Information Management System (LIMS) housed inside the analysis system. The doctor may enter the patient identification which may be transferred to the LIMS. The LIMS may review the inventory database to determine if the test consumables are in stock in the lab room. If the consumables are not in stock, the doctor may place an order for the consumables. If the consumables are in stock, the doctor may confirm the order for the test. The LIMS may send the test order alert to a nurse, phlebotomist, or other sample collector via a sample collection, tracking, and meta data application. The sample collector may check the ordered test information, including the type of sample to be collected and device for collecting the sample. The sample collector may select a suitable time and location or provide time and location options for the patient to select. The LIMS may send a message to the ward where the patient is located regarding the time and location(s) for sample collection. The patient ward may receive the message regarding time and location and may select a time and location and the LIMS may provide the confirmation to the sample collector.

At the scheduled time, the sample collector may arrive at the patient's location and the sample collector may confirm the identity of the patient. The sample collector may select the designated sampling device, which may be a sealed package, and scan the radiofrequency identifier (RFID) tag attached to the package. The RFID information may be matched to records in the LIMS and records of the sample collection packs, including serial number of the sample collection tube or container and information regarding reagents, buffers, other chemicals within the tube or container, sample extraction tool, etc. may be generated. The sample collector may collect the sample and scan the identification number of the sample tube or container using an RFID reader. The identification number may be provided to the LIMS and added to the unique test order entry, which may include the patient identification, test ordered, sample collection package identification, sample container unique identification number, or any combination thereof. The sample collector may collect additional meta data from the patient, including alcohol use prior to sampling, smoking, medication, diet, location from which the sample is taken, or any combination thereof and the meta data may be sent to the LIMS and added to the test record.

### Example 3: System Processing

The collected sample may be delivered to the laboratory. The lab technician or operator may receive the sample and visually verify the contents of the sample. The lab technician may load the sample into a sample holder to load into the system. The LIMS system may prompt the lab control unit to activate the RFID scanner to scan the identification number of the sample tube or container. The identification number(s) may be provided by the RFID scanner to the control unit. The control unit may provide the identification number(s) to the LIMS. The LIMS may match each sample tube identification number to a test record and, from the test record, identify the tests to be run for each sample. The LIMS may determine which sample construction chip and processes will be used for the test. This process may be repeated for all samples loaded into the system.

The LIMS may generate a list of sample identification numbers, identification number of tests for each sample, the sample construction process for each test, and the chips to be used for each test. The LIMS may instruct the control unit to scan the RFID tags of the chips currently loaded within the system. The LIMS may check to determine if the chips for the tests are present within the system and may display, to the laboratory technician, a prompt to load any missing chips. When all chips are present, the LIMS may instruct the control unit to begin processing the sample for construction and testing using the matching chips for the test to be run.

The sample may be provided in or may be transferred to a nucleic acid extraction chip which may lyse any cells in the sample and extract and purify the nucleic acids. The quantity and quality of the extracted and purified nucleic acids may be determined using a built in nucleic acid analyzer. If the extracted and purified nucleic acids do not meet or exceed the quantity and quality standard of the tests ordered extraction and purification of the sample may be repeated until the standards are met. If the extracted and purified nucleic acids meet or exceed the quantity and quality standard of the tests ordered, the extracted and purified nucleic acid may be transferred to a sample construction chip. The LIMS may instruct the control unit to activate electromechanical and chemical processes, such as heating, cooling, sonication, and mixing of purified samples with reagents and markers specific for the protocol of the ordered test for sample construction. The constructed sample may be tested for quality and quantity to verify results are in line with the standards of the specified test. If the results are not in line with the standards of the specific test, sample construction may be repeated. If the results are in line with the standards of the specific test, the constructed sample may be transferred to a sequencer cartridge using robotic liquid handlers and pipettes for sequencing.

A robotic handling sub-system may insert the cartridge into the sequencer and place the relevant reagent bottles needed by the sequencer. Additionally, the robotic handling sub-system may collect a flow cell from the flow cell holder in the lab and insert the flow cell into the sequencer. Once the sample cartridge, flow cell, and reagent bottles are placed inside the sequencer, the control unit may activate the sequencer to commence sequencing. The sequencer may begin sequencing and may provide the raw reads to the in-lab analytics unit.

### Example 4: Dry Lab Processing

Upon receipt of the raw reads, the in-lab analytics unit may begin reconstruction and alignment of the sequencing reads and pattern mating the reads to target genes. The in-lab analytics unit may complete alignment, reconstruction, and pattern matching upon completion of sequencing or shortly thereafter. The in-lab analytics unit may generate a report of the sequenced genes and transmit the report to the LIMS which may transfer the report to the Global Control Center. The Global Control Center may extract select data from the report and transfer the selected data to the knowledgebase and bioinformatics system, which may match the report data and patient profile to existing clinical studies and research papers. The relevant study summaries and references may be attached to the report to generate an appended report. The appended report may be submitted to a second opinion network. The second opinion network may identify the most suitable domain experts and provide a summary of the report to the domain experts with a request to accept the job. An expertise ranking algorithm may select the top two or three domain experts and sent them a full and anonymized report. The selected domain experts may review the report and give their opinion through an application or web interface. The reviewed report may be submitted to the Global Control Unit, which may pass the review and recommendations to the knowledge base and bioinformatics system. The knowledge base and bioinformatics system may provide the full report to the doctor or clinician.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A system for processing a sample, comprising:
an input module configured to accept one or more chips from a user, wherein at least one of said one or more chips comprises said sample;
a processing module configured to transfer said one or more chips from said input module to one or more cartridges, wherein said one or more cartridges are configured to process said sample; and
one or more computer processors operatively coupled to said input module and said processing module, wherein said one or more computer processors are individually or collectively programmed to (i) direct said processing module to transfer said one or more chips from said input module to said one or more cartridges and (ii) direct said one or more cartridges to process said sample.

2. The system of claim 1, wherein said system is configured as a closed system.

3. The system of claim 1, further comprising an alert module configured to monitor said system.

4. The system of claim 3, wherein said alert module alerts a user if said processing module is operating outside of set operating parameters.

5. The system of claim 3, wherein said alert module is configured to alert a user if said system has been opened or otherwise tampered with.

6. The system of claim 1, wherein said system further comprises a tracking module configured to track said sample as it is processed by said system.

7. The system of claim 6, wherein said one or more chips or said one or more cartridges comprise (i) at least one machine readable identifier configured to be tracked by said tracking module as said sample is processed by said system or (ii) at least one human readable identifier.

8. The system of claim 1, further comprising a sequencing unit configured to sequence at least a portion of said sample.

9. The system of claim 1, further comprising one or more detection modules configured to analyze said sample.

10. The system of claim 1, wherein said system is coupled to a cloud server, and wherein said cloud server is configured to permit a user of said system to remotely monitor and control said system.

11. The system of claim 1, wherein said processing module comprises one or more components selected from the group consisting of a liquid handling unit, a pneumatics unit, a temperature control unit, and a transfer unit.

12. The system of claim 1, wherein said processing module is configured to transfer said sample from a first chip of said one or more chips to a second chip of said one or more chips.

13. The system of claim 1, wherein said processing module is configured to (i) transfer a chip of said one or more chips from a first cartridge of said one or more cartridges to a second cartridge of said one or more cartridges or (ii) transfer said sample from said chip to one or more other chips.

14. The system of claim 1, wherein said one or more chips comprise a plurality of ports sealed with a self-sealing material.

15. The system of claim 14, wherein said one or more chips are configured to be fluidically connected using disposable connectors configured to puncture said self-sealing material.

16. (canceled)

17. A system for analyzing a sample, comprising:
a processing module configured to couple to one or more cartridges, wherein, during use, a cartridge of said one or more cartridges comprises at least a portion of said sample;
a detection module comprising at least one sensor configured to analyze said at least said portion of said sample, wherein said at least one sensor comprises a variable pathlength spectrophotometer configured to be in optical communication with at least a portion of said cartridge comprising said at least said portion of said sample; and
one or more computer processors operatively coupled to said processing module and said sensing module, wherein said one or more computer processors are individually or collectively programmed to (i) direct said processing module to couple to said one or more cartridges, (ii) direct said detection module to be in optical communication with said at least said portion of said cartridge comprising said at least said portion of said sample, and (iii) direct said detection module to analyze said sample.

18. The system of claim 17, wherein a pathlength of said variable pathlength spectrophotometer is configured to vary from about 0.01 millimeters (mm) to 0.2 millimeters.

19. The system of claim 17, wherein said system is configured as a closed system.

20. The system of claim 17, further comprising an alert module configured to monitor said system.

21-93. (canceled)

94. A method for analyzing a sample of a subject, comprising:
a. providing a computer server in communication with a user device and an analysis module, wherein said computer server comprises (i) a database for storing test information (ii) a memory for storing a set of software instructions, and (iii) one or more computer processors configured to execute said set of software instructions;
b. receiving one or more input parameters from said user device, wherein said one or more input parameters comprise type of analysis and number of tests to be performed;
c. querying said database to determine one or more testing conditions for performing said analysis of said sample;
d. receiving a status of said analysis module from said analysis module;
e. using said one or more input parameters, said one or more testing conditions, and said status of said analysis module to generate a testing schedule with a minimum testing turnaround time; and
f. providing said testing schedule to said analysis module to perform said analysis of said sample.
